SEQUENCE LISTING (7 sequences)

SEQ ID NO: 1 - DNA, 23 bp, Artificial Sequence
  Upstream primer
ccgcatatggcacggcgagctcg

SEQ ID NO: 2 - DNA, 28 bp, Artificial Sequence
  Downstream primer
gctcaaagatctttgcggcccttcctcg

SEQ ID NO: 3 - RNA, 52 nt, Artificial Sequence
  Secondary Structure of 16S rRNA
acguucccgggccuuguacacaccgccccgucacgucaugaaagucgguaac

SEQ ID NO: 4 - RNA, 42 nt, Artificial Sequence
  Secondary Structure of 16S rRNA - anti-sense
ggugggaucggcgauugggacgaagucguaacaagguagccg

SEQ ID NO: 5 - RNA, 54 nt, Artificial Sequence
  Secondary Structure of half of 23S rRNA
aaacgggcggugguaacuauaaccauccuaagguagcgaaauuccuugucgggu

SEQ ID NO: 6 - protein, 268 aa, Mycobacterium tuberculosis
MARRARVDAELVRRGLARSRQQAAELIGAGKVRIDGLPAVKPATAVSDTTALTVVTDSERAWVSRGAHKLVGALEAFAIAVAGRRCLDAGASTGGFTEVLLDRGAAHVVAADVGYGQLAWSLRNDPRVVVLERTNARGLTPEAIGGRVDLVVADLSFISLATVLPALVGCASRDADIVPLVKPQFEVGKGQVGPGGVVHDPQLRARSVLAVARRAQELGWHSVGVKASPLPGPSGNVEYFLWLRTQTDRALSAKGLEDAVHRAISEGP

SEQ ID NO: 7 - protein, 269 aa, Mycobacterium smegmatis
MARRARVDAELVRRGLARSRQQAAELIGAGRVRIDGMPAAKPATAVSVDANLTVIESDERTWVSRGAHKLIGALDAFGLSVEGRRCLDAGASTGGFTEVLLDRGAAEVVAADVGYGQLAWSLRSDPRVHVLERTNVRDLTPEAIGGPVQMIVADLSFISLATVLPALTACAVRDADIVPMVKPQFEVGKDRVGAGGVVSDPELRIDAVCTVGRKAAALQWHAVDVTASPLPGPSGNVEYFLRLRAETDSPLEGDALEAAVRRAVEEGPQ

(19) United States
(12) Patent Application Publication
(10) Pub. No.: US 2010/0292137 A1
(43) Pub. Date: Nov. 18, 2010

(54) Antibiotics and Methods For Producing Them

(75) Inventors: Stephen Roger Douthwaite, Odense (DK); Shanna Koltzen Johansen, Odense (DK); Gilles Philippus Van Wezel, Oegstgeest (NL)

Correspondence Address: TRASKBRITT, P.C., P.O. BOX 2550, SALT LAKE CITY, UT, 84110, US

(73) Assignee: Mycobics B.V., Leiden (NL)

(21) Appl. No.: 12/223,018

(22) PCT Filed: Jan. 19, 2007
(86) PCT No.: PCT/NL2007/050024

§ 371 Date: Dec. 3, 2008

(30) Foreign Application Priority Data
Jan. 19, 2006 (EP) 06075120.3

(51) Int. Cl. A61K 38/12; C12Q 1/18; C07K 7/64; C12N 1/36; C12N 15/63; C12N 9/10; C12N 1/20; A61P 31/04

(52) U.S. Cl. 514/2.9; 435/32; 530/321; 435/245; 435/471; 435/193; 435/252.1

(57) ABSTRACT

The invention relates to cyclic peptides and methods for producing and using them. Provided is amongst others a method for determining whether a compound comprises antibiotic activity, the method comprising providing a cyclic peptide to a bacterium wherein a 23S rRNA in a 5OS ribosomal subunit does not comprises a 2′ O-methylation at a nucleotide that corresponds to nucleotide C 1920 in E. coli and determining whether said compound inhibits growth of said first bacterium.

The invention relates to the field of cyclic peptide antibiotics and to methods for producing and using cyclic peptides.

Each year about two million people die from tuberculosis (TB), and nine million people become newly infected with its etiological agent, Mycobacterium tuberculosis (Maher and Raviglione, 2005). Incomplete and inadequate antibiotic therapy has led to an increased prevalence of multidrug resistant TB (MDR-TB), which is defined as resistance to two or more first-line antibiotics including isoniazid and rifampicin (Dye et al., 2002). The fall-back position is to treat MDR-TB with second-line antibiotics such as capreomycin (Sharma and Mohan, 2004). Unfortunately, the efficacy of capreomycin is being undermined by its increasing use, which is driving up the frequency of M. tuberculosis strains that are resistant to the drug. The molecular mechanism of capreomycin action has received little attention in recent decades, and a better understanding of this compound is clearly desirable.

Capreomycin and the structurally similar compound viomycin are cyclic peptide antibiotics. Capreomycin and viomycin are particularly active against mycobacteria, and both inhibit growth by blocking protein synthesis on the ribosome (Gale et al., 1981; Vázquez, 1979), although capreomycin is preferred as a therapeutic agent. The drugs interfere with several ribosomal functions including formation of the 30S subunit initiation complex (Liou and Tanaka, 1976) and tRNA translocation from the A to the P site (Modolell and Vázquez, 1977). No crystal structures, which might explain their mode of action, are available for capreomycin or viomycin bound to their ribosomal target. This contrasts with the wealth of structural information on other antibiotics that bind to a single main target situated either on the 30S or on the 50S ribosomal subunit (reviewed by Poehlsgaard and Douthwaite, 2005). Our current knowledge about capreomycin and viomycin binding has been gleaned from other approaches including drug competition (Misumi et al., 1978), ribosome reconstitution (Yamada et al., 1978), cross-resistance (Suzuki et al., 1998; Taniguchi et al., 1997), chemical footprinting (Moazed and Noller, 1987) and translation inhibition studies (Gale et al., 1981; Liou and Tanaka, 1976; Modolell and Vazquez, 1977; Vázquez, 1979).

The recent identification of several M. tuberculosis strains with an unusual type of resistance to capreomycin and viomycin (Maus et al., 2005a) provides a novel angle from which to investigate the mechanism of drug action. Resistance in these strains is conferred by inactivation of the previously uncharacterized tlyA gene. The tlyA phenotype is specific to capreomycin-viomycin with no cross-resistance to other drugs including aminoglycosides. Capreomycin and viomycin susceptibility could be re-established in a resistant tlyA-strain of M. tuberculosis and a non-tuberculous strain of Mycobacterium smegmatis by complementation with an active copy of tlyA (Maus et al., 2005a).

In the present invention, we among others defined the function of TlyA by analyzing the methylation patterns of rRNAs from drug sensitive and resistant strains of M. tuberculosis and M. smegmatis. We show that TlyA is a 2′-O-ribose methyltransferase, and modifies a cytidine on each ribosomal subunit. The 16S and 23S rRNA nucleosides that are methylated by this enzyme were pinpointed precisely using a combination of reverse transcriptase primer extension and Matrix Assisted Laser Desorption/Ionization Time-of-Flight (MALDI-TOF) mass spectrometry. The methylated riboses come into close proximity upon association of 30S and 50S subunits and possibly provide a hydrophobic surface that aids capreomycin-viomycin binding across the subunit interface. Methylation at these riboses is not common in other bacteria, and this is consistent with capreomycin and viomycin being particularly active against mycobacteria. The locations of the ribose methylations delineate the site of capreomycin-viomycin binding, and indicate how these drugs inhibit ribosome function.

The invention therefore in one aspect provides a method for determining whether a compound comprises antibiotic activity comprising providing a compound of formula I to a first bacterium that comprises ribosomes having a 30S and a 50S subunit, wherein a 23S rRNA in said 50S subunit is not methylated at a nucleotide that corresponds to nucleotide C1920 in E. coli, preferably said ribosomes further comprise 30S subunits wherein a 16S rRNA is not methylated at a nucleotide that corresponds to nucleotide C1409 in E. coli, said method further comprising determining whether said compound inhibits growth of said first bacterium.

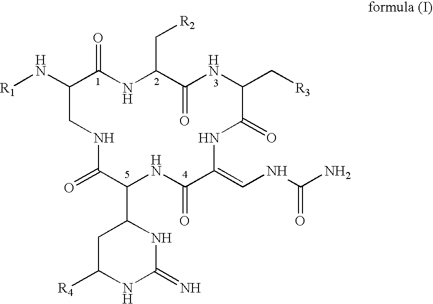

wherein R1 or R3 is a molecule such as a hydrogen, a hydroxy group, an aromatic group, an acylgroup, an amino acid (for example a lysine) or other amide-bond forming molecule, a polyketide, a glycosyl group, an alkyl group, a carbohydrate, a halogenated side chain, or a fatty acid, and R2 or R4 is a hydrogen, a methyl group, a hydroxy group, an acyl group. an amino acid or other amide-bond forming molecule, a fatty acid, a carbohydrate, or a polyketide.

In a preferred embodiment new compounds are generated by combining genes in the biosynthesis cluster for cyclic peptide antibiotics with non-ribosomal peptide synthetases (NRPS) derived from any microorganism (WO 2005/021586). An overview of such NRPS is presented Rausch et al. (Rausch et al., 2005). Example of a biosynthesis cluster for an NRPS for the synthesis of tuberactinomycin-type antibiotics (cyclic peptide antibiotics) is the vio cluster from Streptomyces vinaceus for viomycin biosynthesis (Thomas et al., 2003), and more preferably vioA, vioF, vioG, viol, vioM, and vioO.

Examples of NRPS are found in all actinomycetes, such as SCO7682 and SCO7683 in Streptomyces coelicolor, BAC71354 in Streptomyces avermitilis, PhsB in Streptomyces viridochromogenes, BAD55613, BAD59914, and BAD59915 in Nocardia farcinica, MAP3742 in Mycobacterium avium., AAQ17094-17097 in Amycolatopsis lactamdurans, CAJ34374 in Micromonospora ML1, and also mixed NRPS/polyketide synthases such as AAN85522 in Streptomyces atroolivaceus.

Other sources of NRPS clusters are bacteria, including Pseudomonas, Bacillus, Listeria, brevibacillus, Mesorhizobium, Nostoc, Ralstonia, Xantomonas, and preferably culturable and inculturable microbes from soil or marine origin, and fungi (e.g. Aspergillus and more preferably Aspergillus fumigatus; Gibberella, Hypocrea, Cochliobolus).

Also, adding precursor molecules to a fermentation process will alter antibiotic production (Gastaldo and Marinelli, 2003), and can as such be used for the production of new molecules that fit in the capreomycin or viomycin binding site at the interbridge between the two ribosomal subunits. In another embodiment, biologically synthesized variants can be chemically modified. In this way, a person skilled in the art can synthesize any desired new compound. It is part of the invention that the modifications should be such that they do not increase the size of the molecule to such extent that the antibiotic does no longer fit in the binding site defined in FIG. 9.

Thus in a preferred embodiment the invention provides a method for determining whether a compound comprises antibiotic activity comprising providing a compound, as generated above and/or generated by other combinatorial approaches by manipulating the synthesis of cyclic peptides in bacteria, to a first bacterium that comprises ribosomes having a 30S and a 50S subunit, wherein a 23S rRNA in said 50S subunit is not methylated at a nucleotide that corresponds to nucleotide C1920 in E. coli and determining whether said compound inhibits growth of said first bacterium.

Compounds identified as having antibiotic activity using a method of the invention are typically useful as antibiotics for a large variety of different bacteria. Such compounds are particularly suited antibiotics for the treatment of mycobacterial infections that have developed resistance against capreomycin and/or viomycin.

All bacteria have ribosomes that consist of two subunits. In the present invention these subunits are referred to as the 30S and 50S subunit. These terms identify the subunits for the person skilled in art, although the actual sedimentation characteristics of the subunits in particular cases might be different from the numbers presented.

Antibiotic activity is herein defined as activity at a certain concentration of the antibiotic that inhibits the growth of a tested bacterium when compared to growth of said tested bacterium under comparable circumstances in the absence of said antibiotic. It is preferred that at said concentration, said antibiotic is not toxic to mammalian cells and/or does not inhibit the growth of mammalian cells. Preferably, said mammalian cells are human cells.

The means and methods of the invention and in particular antibiotics of the invention are in one embodiment selected using a first lower eukaryote instead of said first bacterium, and/or a second lower eukaryote instead of said second bacterium. It was found that lower eukaryotes comprise mitochondria that have a similar kind of sensitivity, not necessarily similar amount of sensitivity to the antibiotics of the invention as bacteria specified herein. As a result said mitochondria are at least less functional in the presence of an antibiotic of the invention, leading to at least a reduction in the fitness and/or growth of said lower eukaryote. Thus the invention further provides a use of an antibiotic of the invention for at least reducing the fitness an/or growth of said lower eukaryote. In a preferred embodiment said lower eukaryote comprises a fungus, preferably a yeast or a ascomycete. In another preferred embodiment said lower eukaryote comprises a helminth, preferably a trematode, a cestode or a nematode. Preferred trematodes are Clonorchis, Fasciola and Schistosoma. Preferred cestodes are Taenia and Echinococcus. Preferred nematodes are Trichinella, Strongyloides, Necator and Ancyclostoma. In another preferred embodiment said lower eukaryote comprises a protozoa. Preferred protozoa are Sarcomastigophora, Apicomplexa, Microspora or Ciliophora. Preferred Sarcomastigophora are Amoeba, Tripanosoma or Trichomonas. Preferred Apicomplexa are Plasmodium and Toxoplasma.

In the present invention it was found that bacteria that are normally not very sensitive towards capreomycin or viomycin, can be made more sensitive to the mentioned antibiotics by ensuring that the 23S rRNAs in their ribosomes are methylated at a nucleotide position that corresponds to nucleotide C1920 in Escherichia coli and preferably by also ensuring that their 16S rRNAs in their ribosomes are methylated at a nucleotide position that corresponds to nucleotide C1409 of E. coli. Although ribosomal RNAs are typically well conserved among bacteria, there is some sequence variation. The sequence variation is both in the exact position and the exact base that is present at said position. The exact position of the nucleotide is given relative to the position in E. coli. The corresponding position in another bacterium species can be found according to the E. coli rRNA numbering system (Cannone et al, 2002; Noller 1984) and is used throughout. Sequence variation also occurs in the actual base that is present in said position. In the present invention it is preferred that the base at either the position corresponding to position 23S rRNA C1920 in E. coli or at the position corresponding to position 16S rRNA C1409 in E. coli, or at both positions is a C.

As the present invention identified the three-dimensional spaces where capreomycin and viomycin interfere with the function of the ribosome it has rendered 3D modelling of the interaction of such compounds possible. This knowledge and the knowledge of the methylation dependency of the antibiotic activity can be used to design molecules that resemble the structure of capreomycin and viomycin but that interact differently with the ribosomal groups at that position. This 3D modelling can be used to design compounds with an even higher binding affinity in this position. Alternatively compounds can be produced that exhibit a lower minimum inhibitory concentration (MIC) value, when tested for their antibiotic activity. Thus the invention further provides a method for determining whether a compound inhibits growth of a bacterium, comprising creating a three-dimensional model of a compound and fitting said compound model in a space between the 50S and the 30S subunits where the helix 44 of the 16S rRNA in the 30S subunit meets helix 69 of the 23S rRNA in the 50S subunit in a three-dimensional model of a 30S and a 50S subunit of a ribosome, wherein said fitting comprises establishing interaction between residues of said compound and said subunits in said space, said method further comprises producing said compound or equivalent thereof and contacting said compound or equivalent in vitro with said bacterium, and determining whether growth of said bacterium is at least in part inhibited. Said space in said ribosome for fitting said compound is preferably defined by the nucleotides that surround it.

In a preferred embodiment said space is defined by nucleotides that are at least partially within or touch a sphere of 10 Ångstrom that surrounds the nucleotide that correspond to the position corresponding to position of C1920 in 23S rRNA in E. coli or the position corresponding to position C1409 16S rRNA in E. coli (as shown in FIG. 9). In a preferred embodiment said space, with respect to the position corresponding to position of C1920 in 23S rRNA in E. coli is defined by the three-dimensional position of at least the nucleotides of 16S rRNA of FIG. 9C (column 1) and/or the nucleotides 23S of FIG. 9C (column 2). In a preferred embodiment said space, with respect to the position corresponding to position of C1409 in 16S rRNA in E. coli is defined by the three-dimensional position of at least the nucleotides of 16S of FIG. 9C (column 3) and/or the nucleotides 23S of FIG. 9C (column 4). In a preferred embodiment said space is defined by all of the nucleotides specified in FIG. 9C.

In a preferred embodiment said compound is a cyclic peptide, preferably a tuberactinomycin, a capreomycin, a viomycin or a derivative and/or combination thereof. Cyclic peptides are typically produced by bacteria. As not all bacteria produce all variants of cyclic peptides, it is also possible to produce an equivalent having similar interactions with the modeled ribosome.

A cyclic peptide compound of the invention to be tested in a method of the invention is preferably a compound that was not known for its antibiotic activity prior to the invention. A cyclic peptide compound of the invention to be tested in a method of the invention is preferably a compound that has previously not been tested for antibiotic activity towards mycobacteria.

The site of interaction of capreomycin and viomycin in the ribosome is in a space between the 50S and the 30S subunits where the helix 44 of the 16S rRNA in the 30S subunit meets helix 69 of the 23S rRNA in the 50S subunit (FIG. 9). This space is bordered by structures that include interbridge B2a and interbridge B2b, which comprises, amongst others, parts of helix 44 and helix 45 of 16S rRNA and part of helix 69 of 23S rRNA.

A method of the invention can be used to find compounds that are more effective for bacteria that lack methylation at one or more of the indicated positions in the 16S and 23S rRNA. Thus in a preferred embodiment of the invention said bacterium comprises ribosomes having a 30S and a 50S subunit, wherein a 23S rRNA in said 50S subunit is not methylated at a nucleotide that corresponds to nucleotide C1920 in E. coli. Preferably said bacterium comprises ribosomes wherein a 16S rRNA in said 30S subunit is not methylated at a nucleotide that corresponds to nucleotide C1409 in E. coli. More preferably said bacterium comprises no methylation at both of said positions.

Alternatively, a method of the invention is used to find compounds that are more effective for bacteria that have methylation at one or more of indicated positions in the 16S and 23S rRNA. Thus in another preferred embodiment of the invention said bacterium comprises ribosomes having a 30S and a 50S subunit, wherein a 23S rRNA in said 50S subunit is methylated at a nucleotide that corresponds to nucleotide C1920 in E. coli. Preferably said bacterium comprises ribosomes wherein a 16S rRNA in said 30S subunit is methylated at a nucleotide that corresponds to nucleotide C1409 in E. coli. More preferably said bacterium comprises methylation at both of said positions.

In the present invention it was surprisingly found that antibiotic activity of capreomycin and viomycin was dependent on methylation of two positions in rRNA whereas the binding stoichiometry of the antibiotic to a ribosome was close to or exactly 1:1. Thus one capreomycin or viomycin molecule had at least two interaction points with the ribosome when bound thereto. When one of the positions was methylated whereas the other position was not, antibiotic activity was decreased, albeit at a different level than when both positions were not methylated. Thus in a preferred embodiment said compound in the 3D-modelling method is selected on the basis of the fact that best antibiotic activity is obtained when said compound interacts with the ribosome at both of said positions. Either when the nucleotides at these positions are methylated or when they are not methylated. The fitting of said compound thus preferably further comprises selecting a compound that binding interaction is altered and preferably increased at, at least one but preferably at both of said positions.

With the invention it is now also possible to design in vitro selection systems for selecting compounds that bind more efficiently to either methylated or not methylated nucleotides or both at the indicated positions. Thus in another aspect the invention provides a method for determining whether a compound is capable of inhibiting growth of a bacterium, comprising selecting from a collection of compounds a compound that binds to helix 69 of 23S rRNA and providing said compound to a bacterium to determine whether said compound inhibits the growth of said bacterium. In a preferred embodiment said method comprises selecting a compound from said collection of compounds that interacts with said helix 69, and determining whether said compound inhibits translation in a translation assay. Interaction can for instance be inferred by means of chemical footprinting. Preferably, said 23S rRNA is not methylated at a nucleotide that corresponds to nucleotide C1920 in E. coli. Said method can be extended to further comprise generating a collection of compounds that bind in vitro to said helix 69 and selecting from said generated collection a compound that binds in vitro to helix 44 of 16S rRNA. This can again be done by selecting a compound from said collection of compounds that interacts with said helix 44, and confirming the interaction by means of, for instance, chemical footprinting. Said compounds are preferably tested on a bacterium comprising the same methylation (be it absent or present) as in the helix used for screening. Said in vitro method is preferably performed using entire ribosomes. Thus in a preferred embodiment said bacterium comprises ribosomal 23S rRNA that is not methylated at a nucleotide that corresponds to nucleotide C1920 in E. coli.

Selection of compounds according to the invention is preferably combined with a bacterial assay for antibiotic activity of the selected compound. When the compound is selected on the basis of a methylated nucleotide at the herein above indicated position it is preferred that the bacterium used in the assay comprises the same methylation and vice versa, when the compound is selected on the basis of a non-methylated nucleotide at the herein above indicated position it is preferred that the bacterium used in the assay is not methylated at that position. As capreomycin and viomycin have a much higher antibiotic activity on bacteria having ribosomes comprising rRNAs that are methylated nucleotides at said positions, it is preferred to verify whether a compound selected by a method of the invention comprises the same pattern of antibiotic activity, or an altered one. Thus in a preferred embodiment the invention provides a method of the invention further comprising determining whether said compound inhibits growth of a second bacterium comprising the opposite type of methylation pattern when compared to said first bacterium. In other words, when said first bacterium comprises ribosomes with a 23S rRNA that is methylated at the indicated position, said second bacterium preferably comprises the opposite thereof, i.e. 23S rRNA that is not methylated at the indicated position. The same holds true for methylation on 16S rRNA. And the other way around, when said first bacterium comprises ribosomes with a 23S rRNA that is not methylated at the indicated position, said second bacterium preferably comprises the opposite thereof, i.e. 23S rRNA that is methylated at the indicated position. Again, the same holds true for methylation on 16S rRNA. In a preferred embodiment said second bacterium comprises ribosomes having a 30S and a 50S subunit, wherein a 23S rRNA in said 50S subunit is methylated at a nucleotide that corresponds to nucleotide C1920 in E. coli. Preferably, said second bacterium comprises ribosomes wherein a 16S rRNA in said 30S subunit is methylated at a nucleotide that corresponds to nucleotide C1409 in E. coli. A preferred way of ensuring that said first or said second bacterium comprises methylation of said rRNAs at the indicated positions is to use a bacterium that comprises a tlyA gene. Preferably a bacterium is used that not naturally contains a tlyA gene. Thus in a preferred embodiment the invention provides a method of the invention wherein said first bacterium is a bacterium lacking a functional tlyA gene. Preferably because said bacterium is naturally devoid of a functional tlyA gene. In a preferred embodiment said second bacterium is a bacterium comprising a functional tlyA gene. Preferably, said second bacterium is a bacterium that is naturally devoid of a functional tlyA gene, and said bacterium is transformed with a functional recombinant version of the said tlyA gene. In a preferred embodiment said first and/or said second bacterium is E. coli or Mycobacterium smegmatis. In another preferred embodiment said first and/or said second bacterium is of the class of Actinobacteria, preferably of the order Actinomycetales and more preferably of the genus Streptomyces. In a particularly preferred embodiment said bacterium is not a Mycobacterium. Mycobacteria are not easy to grow and to manipulate in vitro. In another embodiment, said first and/or said second bacterium is a Mycobacterium, preferably Mycobacterium tuberculosis and Mycobacterium smegmatis. Sometimes it is preferred to use a Mycobacterium as these are or closely resemble pathogenic variants.

When in the present invention mention is made of a nucleotide C1409 in 16S rRNA or a nucleotide C1920 in 23S rRNA that is not methylated, it is meant that said nucleotide does not comprises a tlyA protein mediated type of methylation. The nucleotide can, of course comprise other modifications, such as but not limited to methylation at other sites on said nucleotide. When in the present invention mention is made of a nucleotide C1409 in 16S rRNA or a nucleotide C1920 in 23S rRNA that is methylated, it is meant that said nucleotide comprises a tlyA protein specific methylation. The nucleotide can, of course further comprise other modifications, such as but not limited to methylation at other sites on said nucleotide. A tlyA protein specific methylation is a 2′ O-methylation.

Cyclic peptide antibiotics are typically produced by bacteria of the class Actinobacteria. With the identification of the genes involved with the production of cyclic peptides it has become possible to use these genes in combinatorial approaches to generate novel cyclic peptides that have, as yet, no counterpart in nature. In the present invention one or more of these combinatorial approaches are used to generate a collection cyclic peptides that can be used in a selection method of the invention. Thus the invention further provides a method for determining whether a compound comprises antibiotic activity comprising generating a collection of related compounds through combinatorial biosynthesis of cyclic peptides, preferably a collection of capreomycins, tuberactinomycins or viomycins or derivatives or a combination thereof, and determining whether a compound from said collection inhibits the growth of a first bacterium that comprises ribosomes having a 30S and a 50S subunit, wherein a 23S rRNA in said 50S subunit is not methylated at a nucleotide that corresponds to nucleotide C1920 in E. coli. Preferably, said ribosomes further comprise 16S rRNA in said 30S subunit that is not methylated at a nucleotide that corresponds to nucleotide C1409 in E. coli. Again, this method can be further extended by determining the antibiotic activity of said compound on a second bacterium having an alternate methylation pattern. Thus in a preferred embodiment said method further comprises determining whether said compound inhibits growth of a second bacterium comprising ribosomes having a 30S and a 50S subunit, wherein a 23S rRNA in said 50S subunit is methylated at a nucleotide that corresponds to nucleotide C1920 in E. coli. Said second bacterium preferably comprises ribosomes wherein a 16S rRNA in said 30S subunit is methylated at a nucleotide that corresponds to nucleotide C1409 in E. coli. In a preferred embodiment said combinatorial biosynthesis of cyclic peptides comprises combinatorial biosynthesis of capreomycins, tuberactinomycins, viomycins or derivatives or combinations thereof. Preferably, said combinatorial biosynthesis of capreomycins, tuberactinomycins, viomycins or derivatives or combinations thereof comprises providing a bacterium that produces said capreomycin, tuberactinomycin, viomycin or derivative or combination thereof, and altering the biosynthesis pathway thereof. In one embodiment said bacterium comprises a capreomycin, tuberactinomycin and/or viomycin biosynthesis pathway as described in Thomas et al (2003), WO2005/021586 and/or U.S. Pat. No. 2,828,245. In a preferred embodiment said bacterium producing a cyclic peptide is of the class of Actinobacteria, preferably of the order Actinomycetales and more preferably of the genus Streptomyces. In a preferred embodiment said first and said second bacterium are E. coli or of the Actinobacteria, preferably of the order Actinomycetales and more preferably of the genus Streptomyces. Preferably, said first and said second bacterium belong to the same genus, more preferably to the same species.

Compounds that are selected by a method of the invention can be provided with additional features to provide additional properties. This can be done prior to or after a method of the invention. A preferred modification is a modification that stimulates membrane penetration of said compound. Thus in a preferred embodiment a method of the invention further comprises providing said compound with a moiety that stimulates membrane penetration, i.e. a membrane penetration moiety. In a preferred embodiment said moiety comprises a cell penetrating peptide.

In yet another aspect the invention provides a compound obtainable by a method of the invention. Further provided is a composition comprising a compound of the invention. Compounds such a capreomycin, viomycin and equivalents thereof have antibiotic activity against bacteria comprising methylated rRNA at the indicated positions. Capreomycin and viomycin are currently used to treat mycobacterial infections. A problem is however, the occurrence of resistance to these antibiotics. The present invention provides a new class of antibiotics that exhibit increased antibiotic activity on bacteria having no methylation of rRNA at the indicated positions. In a preferred embodiment of the invention, these compounds are used to treat infection with bacteria comprising no-methylation at rRNA at the indicated positions. In a particularly preferred embodiment, the invention provides a composition comprising both said compound and capreomycin, viomycin or an equivalent thereof. Such a composition can advantageously be used to treat mycobacterial infections and thereby circumvent the problem of resistance to said compounds on the basis of the presence or absence of methylation of rRNA on the indicated positions. This is because bacteria are confronted with antibiotics that are effective against bacteria comprising methylation at the indicated positions and bacteria that do not comprise said methylation. Thus in a preferred embodiment a composition of the invention further comprises capreomycin, viomycin or a functional equivalent thereof.

Microorganisms can acquire resistance to antibiotics in various ways. Resistance to cyclic peptide antibiotics is acquired, among others, through mutation or modification of ribosomal RNA. Antibiotics that are selected through a method of the invention are no different in this respect than the classical antibiotics. Thus the use of antibiotics of the invention will select for microorganisms that are, or have acquired resistance to the antibiotic. Thus the present invention further provides a microorganism that has acquired resistance to an antibiotic of the invention. The invention further provides a ribosome and a bacterium comprising said ribosome comprising a mutation in helix 44 of the 16S rRNA together with a mutation in helix 69 of the 23S rRNA other than the nucleotide that corresponds to nucleotide C1920 in the 23S rRNA of E. coli. The invention further provides a ribosome and a bacterium comprising said ribosome comprising a mutation in helix 69 of the 23S rRNA together with a mutation in helix 44 of the 16S rRNA other than the nucleotide that corresponds to nucleotide C1409 in E. coli in the 16S rRNA of E. coli.

In yet another embodiment, the invention provides a microorganism comprising said ribosome. The present invention further provides modelling a mutation that confers resistance to an antibiotic of the invention and designing a compound derived from said antibiotic that is not affected by said resistance conferring mutation.

In one embodiment the invention provides the use of a compound or a composition of the invention, for the preparation of a medicament for at least in part inhibiting growth of a bacterium. Preferably said bacterium is a Gram positive bacterium. Preferably said bacterium is an actinomycete. In another preferred embodiment said bacterium is a Mycobacterium. Preferably said bacterium comprises a C as the nucleotide that corresponds to nucleotide C1920 in the 23S rRNA of E. coli. and a C as the nucleotide that corresponds to nucleotide C1409 in E. coli in the 16S rRNA of E. coli.

The invention further provides a pharmaceutical composition comprising a compound or a composition of the invention, and optionally a pharmaceutically acceptable carrier.

The invention further provides the use of a compound or a composition of the invention, for at least in part inhibiting translational activity by a ribosome of a bacterium. Also provided is a use wherein said translational activity is inhibited by interference with tRNA translocation from the A site to the P site.

The invention also provides a method for producing a modified capreomycin or viomycin, comprising providing capreomycin or viomycin with a substituent that enhances interaction of said antibiotics with 23S rRNA in a ribosome that is not methylated at a nucleotide that corresponds to nucleotide C1920 in E. coli and/or with 16S rRNA in said ribosome that is not methylated at a nucleotide that corresponds to nucleotide C1409 in E. coli, to enhance binding thereto of said modified capreomycin or viomycin. Such a substituent is preferably a hydrophilic substituent.

In yet another embodiment, the invention provides a method for conferring antibiotic sensitivity to a bacterium that is resistant to said antibiotic, comprising inserting a tlyA gene in the genome of said bacterium. In a preferred embodiment said antibiotic comprises capreomycin, viomycin or an equivalent thereof. In a preferred embodiment said bacterium is not a Mycobacterium. In a preferred embodiment said bacterium is E. coli or a bacterium of the class of Actinobacteria, preferably of the order Actinomycetales and more preferably of the genus Streptomyces. Preferably said bacterium comprises a C as the nucleotide that corresponds to nucleotide C1920 in the 23S rRNA of E. coli. and a C as the nucleotide that corresponds to nucleotide C1409 in E. coli in the 16S rRNA of E. coli.

The invention further provides a method for producing an antibiotic of the invention comprising culturing a bacterium producing said antibiotic and harvesting culture medium from said culture. In a preferred embodiment said bacterium is of the class of Actinobacteria, preferably of the order Actinomycetales and more preferably of the genus Streptomyces. Said bacterium can be selected using a method of the invention. In a preferred embodiment said bacterium is Streptomyces vinaceus or Streptomyces capreolus (Skinner and Cundliffe, 1980).

In yet another embodiment the invention provides a bacterium provided with a tlyA gene. In a preferred embodiment said bacterium is not a Mycobacterium. In a preferred embodiment said bacterium is E. coli. The invention further provides the use of a bacterium provided with a tlyA gene for selecting a compound with the antibiotic activity. The invention further provides a use of a tlyA gene or a protein encoded thereby for increasing the antibiotic activity of capreomycin, viomycin or equivalent thereof on a bacterium. Preferably said bacterium comprises a C as the nucleotide that corresponds to nucleotide C1920 in the 23S rRNA of E. coli and a C as the nucleotide that corresponds to nucleotide C1409 in E. coli in the 16S rRNA of E. coli.

As used herein, a tlyA gene sequence comprises a sequence encoding a protein as depicted in FIG. 8 or a functional equivalent thereof. A functional equivalent of a tlyA gene encodes essentially the same protein as a tlyA gene or encodes a functional equivalent of said TlyA protein. Such sequence may differ substantially from the sequence of a tlyA gene as the genetic code is degenerate. Such sequence may be optimised for the specific codon usage of the organism it is produced in. A TlyA protein comprises an amino acid sequence of FIG. 8; however, the sequence of the TlyA protein may vary at nonessential sites while having the same overall tertiary fold. A functional equivalent of a TlyA protein comprises the same methylation properties in kind, not necessarily in amount. In a preferred embodiment said tlyA gene equivalent is a tlyA gene of a tlyA containing bacterium other than a mycobacterium. In a preferred embodiment said tlyA gene equivalent comprises a tlyA gene of Serpulina hyodysenteriae, Streptomyces coelicolor, Thermus thermophilus and Geobacillus stereothermophilus or a functional equivalent thereof encoding the same TlyA protein in kind.

The invention further provides an actinomycete wherein the tlyA gene is functionally deleted. In a preferred embodiment said deletion comprises at least 10 amino acids. Preferably, said deletion comprises the entire coding region. Preferably said deletion comprises an in frame deletion. The deletion preferably leaves other genes and other open reading frames intact. Thus in a preferred embodiment said deletion leaves the expression and integrity of the downstream located genes intact. In a preferred embodiment said actinomycete is a Streptomyces. In a particularly preferred embodiment said Streptomyces comprises S. coelicolor.

Inactivation of the tlyA gene in M. tuberculosis confers resistance to the ribosome-targeting drugs capreomycin and viomycin (Maus et al., 2005a). We show here that the active tlyA gene encodes an enzyme specific for 2′-O-methylation of riboses in rRNA, and that lack of these methylations results in drug resistance. Resistance to ribosome-targeting drugs is generally associated with addition of methyl groups to the rRNA rather than their loss (Cundliffe, 1989; Douthwaite et al., 2005). TlyA thus belongs to an exclusive group of methyltransferases that confer antibiotic resistance by losing their function—the only other characterized group member being KsgA (Heiser et al., 1972; Van Buul et al., 1983). KsgA and TylA are S-adenosylmethionine-dependent methyltransferases and both modify two nucleotides within rRNA, although they differ in their substrate recognition and their sites of nucleoside modification. KsgA catalyzes the N⁶, N⁶-dimethylations of the two adenosines A1518 and A1519 in the loop of 16S rRNA helix 45 (FIG. 1A), and loss of these methylations confers kasugamycin resistance (Helser et al., 1972; Van Buul et al., 1983). TlyA methylates the ribose at nucleotide C1409 in helix 44 of 16S rRNA and the ribose at nucleotide C1920 in helix 69 of 23S rRNA (FIG. 1). TlyA is the first reported case of a bacterial methyltransferase that modifies specific nucleotides within the rRNAs of both ribosomal subunits. The identification of the TlyA-methylations helps clarify a number of previous observations concerning the mode of action of capreomycin and viomycin, and why these drugs are particularly active against mycobacteria.

The two sites of TlyA modification come into close proximity when the 30S and 50S subunits associate to form 70S ribosome couples (FIG. 7). Subunit association has long been known to involve rRNA interactions (Mitchell et al., 1992), and the recent crystal structures of 70S ribosomes from Thermus thermophilus (Yusupov et al., 2001) and E. coli (Schuwirth et al., 2005) clearly show that association occurs via interbridge structures, which for the most part involve direct interaction between the 16S and 23S rRNAs. Interbridge B2a is formed between the loop of 23S rRNA helix 69 and 16S rRNA helix 44 in the region of nucleotide 1409; interbridge B2b is formed immediately adjacent by contact between the helix 69 backbone at C1920 and the loop of the 16S rRNA helix 45. The E. coli ribosome crystals diffract to higher resolution (3.5 Å), and contain two ribosome conformers in which the riboses of C1409 and C1920 are 19.8 Å and 21.4 Å apart on opposite sides of the B2a interbridge (Schuwirth et al., 2005). The B2b bridging interactions noted in the Thermus structure are fractionally out of contact range in the E. coli structure, although both models place the C1920 ribose of 23S rRNA within 10 Å of the A1518 methylated base in 16S rRNA (FIG. 7).

The B2a and B2b interbridges have been shown by chemical probing studies to be essential not only for subunit association (Maivali and Remme, 2004; Merryman et al., 1999a; Merryman et al., 1999b), but also for the interaction of tRNAs with the ribosome (Moazed and Holler, 1989a, 1990). The B2a interbridge is at the geometric centre of the subunit interface, and marks the border between the ribosomal A and P sites (FIG. 7). The anticodon loop of the A-site tRNA is close to 16S rRNA nucleotide C1409 (Moazed and Noller, 1990; Valle et al., 2003a; Yusupov et al., 2001), while the top of the P-site tRNA anticodon stem is closer to the ribose of C1920 (Joseph et al., 1997; Moazed and Noller, 1989a; Yusupov et al., 2001). Disruption of the interbridge structures or attaching a ligand to interbridge B2a could be expected to interfere with tRNA binding and/or tRNA translocation from the A site to the P site.

According to earlier studies, viomycin (and, thus, presumably capreomycin) inhibit formation of the translational initiation complex (Liou and Tanaka, 1976), a process that involves placement of the initiator tRNA into the P site. Viomycin also inhibits later stages of translation by blocking translocation of the peptidyl-tRNA from the A to the P site (Liou and Tanaka, 1976; Modolell and Vazquez, 1977). Translocation is blocked after peptide bond formation has occurred and without directly preventing elongation factor G from hydrolyzing GTP (Modolell and Vazquez, 1977; Vázquez, 1979). Reinterpreting these observations in terms of the tRNA hybrid-site model (Moazed and Noller, 1989b), it appears that viomycin confines the peptidyl tRNA to the A/P state, where the 3′-end of the tRNA has moved to the P site on the 50S subunit and has accepted the peptide chain, while the anticodon end of the tRNA remains locked in the A site on the 30S subunit. Viomycin could block the translocation process in at least two ways: by acting as a physical barrier to the passage of the tRNA from the A/P site or, alternatively, by enhancing the association of the ribosomal subunits (Yamada and Nierhaus, 1978) and thereby impairing their relative movement required for translocation. Both putative modes of inhibitory action are consistent with viomycin (and capreomycin) binding to the ribosomal interface at the interbridge region that coincides with the positions of TlyA methylation.

Capreomycin and viomycin are particularly effective against mycobacteria, and capreomycin is presently widely used as a second-line agent against tuberculosis. Homologues of tlyA are absent from many genera of bacteria, and this corresponds with a generally low level of susceptibility to capreomycin and viomycin. For instance, E. coli and other enterobacteria lack a tlyA homologue and the ensuing rRNA methylations (Rozenski et al., 1999 and our unpublished data) and, consistent with this, E. coli is relatively resilient towards capreomycin and viomycin even when its cell membrane is compromised (Table 2). Upon expression of recombinant tlyA in E. coli, the rRNA becomes methylated at the same two ribose sites as in mycobacteria (data not shown), and this is accompanied by a distinct and specific increase in the sensitivity towards capreomycin and viomycin (Table 2). TlyA methylation thus clearly enhances the activity of capreomycin and viomycin and is the cause, at least in part, of the efficacy of these drugs against mycobacteria.

The methylation sites reported here fit well with the earlier observation that changes to the rRNA in either subunit confers viomycin resistance (Yamada et al., 1978). The specific importance of C1409 and neighboring 16S rRNA nucleotides for capreomycin and viomycin activity has previously been established by a series of mutations in this region (Maus et al., 2005a, b; Taniguchi et al., 1997), which support the direct involvement of TlyA methylation at C1409 in drug binding. Of particular interest is the rrl ΔA1916 mutant where the C1409 methylation is maintained while the C1920 methylation is lost, together with the knowledge of the present invention this mutant thus firmly establishes the involvement of 23S rRNA helix 69 in capreomycin-viomycin activity. It remains unclear whether the lack of C1920 methylation and/or the deleted 1916 nucleotide is conferring the resistance phenotype in the rrl ΔA1916 strain, although the higher resistance (compared to the tlyA mutants) indicates that the A1916-deletion disturbs drug binding in a more direct manner than merely by blocking TlyA methylation. It should be noted that we found no support for the claim that loss of N-1 methylation at G745 confers viomycin resistance (Gustafsson and Persson, 1998) (Table 2); neither can we reconcile our findings with the viomycin footprints at 23S rRNA nucleotides U913/G914 obtained using a large molar excess of drug (Moazed and Noller, 1987). The G745 and U913/G914 regions are both located quite distant from nucleotide 1920 in the 23S rRNA tertiary structure.

Questions remain as to how the TlyA methylations enhance the activity of capreomycin and viomycin, and whether both methylations contribute equally to drug binding. The drugs possess numerous hydrophobic edges on their composite ring and alkyl chain structures (Gale et al., 1981; Vázquez, 1979), which could be envisaged to interact with hydrophobic patches provided by the TlyA methylations. The 19 Å to 21 Å distance between the two methylations, deduced from the two highly resolved E. coli ribosome structures (Schuwirth et al., 2005), is just within span of the largest variants of the drugs. These distances quite feasibly reflect just two of the physiological viable conformations that arise from the relative movement of the subunits during translation (Valle et al., 2003b). Presumably, one ribosome conformation provides an optimal spacing between nucleotides C1409 and C1920 that is bound and locked by viomycin and capreomycin.

### EXAMPLES

### Strains Used in this Study

The mycobacterial strains used in this study are listed in Table 1, and comprise wild-type and capreomycin/viomycin resistant isolates of M. tuberculosis as well as laboratory strains of the non-tuberculous species M. smegmatis. The resistant M. tuberculosis isolates arose from two virulent wild-type strains (H37Rv and Beijing D3); resistant M. smegmatis strains were isolated from the wild-type strain LR222. Growth of all the wild-type strains is completely inhibited by capreomycin and viomycin concentrations of less than 10 μg/ml. The tlyA mutants P2U, 315-A, C-202 and C-211 have capreomycin and viomycin MICs of 20 to 40 μg/ml, while remaining susceptible to the aminoglycosides kanamycin and amikacin (Maus et al., 2005a). No noticeable differences in growth rate were observed between the wild-type strains and the tlyA mutant strains when grown in medium without capreomycin.

One of the spontaneous capreomycin-resistant mutants, C-401, is distinctly different from the other isolates. C-401 has wild-type tlyA and rrs (16S rRNA) sequences, but possesses a deletion in the rrl gene corresponding to loss of 23S rRNA nucleotide A1916 (E. coli numbering is used throughout). The rrl ΔA1916 mutant is resistant to capreomycin (MIC>160 μg/ml) and viomycin (MIC>80 μg/ml) while remaining susceptible to kanamycin and amikacin. In medium without drug there is no discernible difference in the growth rate of the rrl ΔA1916 mutant and the wild-type parent strain; furthermore, low concentrations of capreomycin (at 10 μg/ml) do not noticeably slow the growth rate of the rrl ΔA1916 mutant.

Screening of rRNA from Wild Type and Mutant Strains

We selected seven rRNA regions for our initial screening, covering the majority of known modification sites (Rozenski et al., 1999), which tend to occur in clusters (Brimacombe et al., 1993) within conserved and functionally important ribosomal regions (Gutell et al., 1994). Three regions were in the 16S rRNA and included the sequences around A1408 and G1491, where mutations have been shown to be associated with resistance to capreomycin (Maus et al., 2005a; Taniguchi et al., 1997), and the highly modified A1518 region (FIG. 1). Four modified regions were screened in 23S rRNA: these included the methylated loop around G745 (Gustafsson and Persson, 1998; Liu and Douthwaite, 2002a); the pseudouridylated helix 69 containing A1916 (del Campo et al., 2004); the A2058 region that is an important antibiotic target (Schlunzen et al., 2001; Tu et al., 2005); and the U2552 region, which is methylated by RrmJ (Caldas et al., 2000). These seven regions in the rRNAs of wild-type and mutant mycobacterial strains (Table 1) were initially investigated by primer extension with reverse transcriptase, enabling us to screen sequences of 200 to 300 nucleotides from each primer.

Differences in rRNA Methylation

Differences between the wild-type and mutant primer extension patterns were seen at C1409 in 16S and at C1920 in 23S rRNA. No differences in the methylation patterns were observed in the 16S rRNA 1518 region, nor in the 23S rRNA 745, 2058 and 2552 regions (data not shown), and none of these sequences were subjected to further analysis. The primer extension analysis of the 1491 region in 16S rRNA was ambiguous due to primer hybridization problems; however, this region was later ruled out as a TlyA target using mass spectrometry.

Nucleotides C1409 and C1920 were analysed more closely using primers that hybridize immediately adjacent to these sites (FIG. 2). Extension on the rRNAs from all the wild-type M. tuberculosis and M. smegmatis strains was preferentially terminated at C1409 and C1920 with progressively decreasing dNTP concentrations, suggesting that these were possible 2′-O-methylation sites. Extension on the rRNAs from the mutant tlyA strains showed no stops at C1409 or C1920 indicating loss of methylation. Intriguingly, the ΔA1916 rrl mutant (C-401) retained the C1409 methylation, but lost the methylation at C1920 (FIG. 2). All the rRNAs were subjected to further analysis by mass spectrometry to verify the positions of the modifications, and to determine whether these were indeed ribose methylations.

Mass Spectrometry Analysis of the rRNA

The masses of RNA oligonucleotides can be accurately measured (to within 0.2 Da) by MALDI-TOF mass spectrometry. Modifications such as methylations can thus be readily identified in 16S and 23S rRNAs after digestion with specific RNases to yield oligonucleotides of suitable size. However, digestion of these large rRNA molecules results in numerous oligonucleotides with similar or identical masses, and produces a complex mass spectrum. To circumvent this problem, we selected specific rRNA regions of about 50 nucleotides by hybridizing to complementary oligodeoxynucleotides (FIG. 1). The hybridized regions were isolated and digested with RNase A or RNase T1, and gave rise to unambiguous mass spectra in which the sites of rRNA modification could be readily identified.

Helix 44 in 16S rRNA

The nucleotide sequences from 1377 to 1427 and 1461-1511 in helix 44 of 16S rRNA (FIG. 1) were isolated by hybridization, and then cleaved with the guanosine-specific RNase T1. In the 1461-1511 region, there were no differences between the mass spectra derived from the wild-type and the mutant rRNAs (data not shown). In the 1377 to 1427 region, however, the wild-type spectrum peak at m/z 1637 was shifted to m/z 1623 in the spectra from all the tlyA mutants (FIG. 3). Both peaks correspond to the 1406-1410 sequence (5′-UCACGp), which would have a theoretical m/z of 1609 in the unmodified state and thus contains two methyl groups in the wild-type strains, but only one methyl group in the tlyA mutants. The rrl ΔA1916 mutant retained both of these 16S rRNA methylations (data not shown).

The m/z 1623 and m/z 1637 peaks were selected and fragmented further by collision-induced dissociation using MALDI quadrupole-TOF tandem MS (FIG. 4), revealing their sequence as well as the locations of the methylations. The one methyl group is present in both ions, and is attached to the base of G1410. The second methyl group is on the 2′-O-ribose position of nucleotide C1409 in the wild-type rRNAs (m/z 1637 ion), and is missing from the tlyA mutants (m/z 1623 ion). The ion fragments derived from the rrl AA1916 mutant (data not shown) matched the wild-type pattern in this 16S rRNA region.

Helix 69 in 23S rRNA

The 23S rRNA sequence encompassing helix 69 (nucleotides 1899 to 1951, FIG. 1) was isolated from each of the mycobacterial rRNAs (Table 1). After RNase T1 cleavage, MS analyses revealed a peak at m/z 6065 arising from all the wild-type rRNAs, whereas the rRNAs from tlyA mutants produce a peak at m/z 6051 (data not shown). Both peaks correspond to the 23S rRNA sequence 1911 to 1929, and the 14 Da difference in mass reflects the presence or absence of a methyl group. The methylation site was localized by digesting the 1899-1951 rRNA sequences to smaller fragments using RNase A, which specifically cleaves after pyrimidines. The wild-type spectra showed a signal at m/z 1301 (FIG. 5A), corresponding to the methylated fragment 5′-AACCp (A1918-C1921). The lack of cleavage between the two pyrimidines is consistent with a methyl group on C1920 blocking RNase A cleavage after this nucleotide. No spectral peak at m/z 1301 was obtained from the rrl ΔA1916 strain or from any of the tlyA mutants (FIG. 5B). In these spectra, cleavage of the RNAs after an unmethylated C1920 gives rise to the trinucleotide 5′-AACp (A1918-C1920) that runs in the peak at m/z 982 together with a fragment from another region of the sequence.

The exact position of the methyl group in the wild-type rRNAs was verified by MALDI quadrupole-TOF tandem MS analysis of the m/z 1301. fragment. From the fragmentation pattern (FIG. 6) the site of methylation could be unambiguously assigned to the C1920 ribose.

### Drug Contacts Across the Subunit Interface of the 70S Ribosome

Within the 50S subunit of the 70S ribosome, the drugs most notably make interactions between 23S rRNA nucleotides 1900 and 1930, which include helix 69 and its flanking regions. This is evident from the hydroxyl radical footprint showing drug protection around nucleotides C1920 and G1930 (FIG. 10), and from DMS footprints showing several protections, including A1913 in the loop of helix 69 (FIG. 11). In all cases, no comparable footprints are seen after addition of the drugs to the purified 50S subunit.
In the 30S component of 70S ribosomes, drug footprints were also evident, and these were missing when purified 30S subunits were tested for drug binding. On 70S ribosomes, the drugs make a clear interaction with 16S rRNA protecting nucleotide A1408 from modification by DMS (FIG. 12); the drugs do not afford such protection when incubated with purified 30S subunits.
It can be concluded that binding interactions between the cyclic peptide antibiotics capreomycin and viomycin and the ribosome occur only when the ribosomal subunits are associated into 70S couples. These drug interactions do not occur on the individual subunits. In the associated 70S ribosome, helix 69 in 23S rRNA and nucleotide A1408 of 16S rRNA face each other across the ribosomal interface to form the binding site for the cyclic peptide antibiotics capreomycin and viomycin.

### Other Footprint Effects Outside the Immediate Region of Drug Contact

Capreomycin-viomycin binding to 70S ribosomes stabilizes the interaction between the two subunits. This is evident in footprint protections that lie outside the immediate vicinity of capreomycin-viomycin contacts. The majority of these effects are at or close to the subunit interface, which suggests that capreomycin-viomycin binding brings the subunits into closer contact and thereby reduce access of the interface to the chemical reagents. These effects are illustrated by hydroxyl radical footprints for regions of 23S rRNA (FIGS. 13 & 14) and for both hydroxyl radical and DMS footprints in 16S rRNA (FIGS. 15 & 16). FIG. 17 shows a summation of the footprint effects.
TlyA Proteins from Different Species Methylate rRNA In Vivo

Homologues of the M. tuberculosis and M. smegmatis tlyA gene were amplified by PCR from Serpulina hyodysenteriae (formerly Brachyspira hyodysenteriae), Streptomyces coelicolor, Thermus thermophilus and Geobacillus stereothermophilus (formerly Bacillus stereothermophilus), and were cloned behind the lac promoter in an E. coli expression vector (see below). The origins of the tlyA genes are listed in Table 4.

The rRNAs of E. coli are not naturally methylated at the TlyA targets (C1409 in 16S, and C1920 in 23S rRNA). Induction of the recombinant tlyA genes led to methylation of the E. coli rRNAs in vivo (FIG. 18). Expression of all the recombinant TlyA enzymes led to methylation of 23S rRNA C1920. Nucleotide C1409 in 16S rRNA was also methylated by the recombinant M. tuberculosis, M. smegmatis, S. coelicolor and G. stereothermophilus TlyA enzymes; the enzymes from S. hyodysenteriae and T. thermophilus did not methylate C1409.
Expression of tlyA Homologues Increase Susceptibility to Capreomycin-Viomycin
Expression of the different tlyA genes rendered E. coli more susceptible to the cyclic peptide drugs. Minimal inhibitory concentrations (MICS) for the different recombinants are listed in Table 6. Expression of tlyA lowered the MIC of capreomycin required to inhibit cell growth. The Thermus and Serpulina tlyA recombinants demonstrate that methylation at 23S rRNA nucleotide C1920 is enough to increase drug sensitivity. Thus, loss of this single methylation must confer mild resistance. Expand the point that resistance can be conferred by disruption of either the 16S rRNA or the 23S rRNA drug contact site (the first by mutation at A1408/C1409, the second by loss of methylation).
Activity of tlyA in Streptomyces

### Capreomycin Resistance of Wild-Type Streptomyces Species

To obtain insight as to how resistant wild-type Streptomyces species are to capreomycin, the well-known streptomycetes S. coelicolor M145, S. griseus NRRL B2682, S. lividans 1326 and S. avermitilis MA4630 were streaked on SFM agar plates with different concentrations of capreomycin. This showed that S. avermitilis and S. coelicolor had a MIC value between 1-5 μg/ml capreomycin, while the MIC value of S. griseus and S. lividans lay between 5 and 10 μg/ml capreomycin (FIG. 19). At sub-lethal concentrations, development was inhibited and antibiotic production was enhanced, exemplified by the strong overproduction of the blue-pigmented antibiotic actinorhodin by S. coelicolor at capreomycin levels above 1 μg/ml, most likely as a stress response.
Capreomycin Resistance of S. coelicolor Relates to Spontaneous Mutations in tlyA
The data on Mycobacterium and the high degree of conservation of TlyA in many different bacteria strongly suggest that TlyA should directly relate to capreomycin sensitivity/resistance in all bacteria with a tlyA gene. To obtain more insight in the relationship between TlyA activity and capreomycin resistance in actinomycetes we set out to obtain spontaneous capreomycin resistant colonies. For this, around 10⁹ colony forming units (cfu) of S. coelicolor M145 were streaked on SFM agar plates containing 25 μg/ml capreomycin. All colonies obtained from this first selection experiment were re-streaked on the same media, and stably resistant colonies were selected. In this way, 17 colonies were obtained that were significantly more resistant to the antibiotic than the original strain (FIG. 20). These were designated CAP1-17. Interestingly, there was a difference in the degree of resistance, and the different classes could roughly be divided in mutant strains with low resistance (CAP5 and 6), medium resistance (CAP 1-4, 7-9, 14) and high resistance (CAP10-13, 15-17) to capreomycin.

Conceivably, the mechanisms that most readily lead to capreomycin resistance through a single mutation should be (1) mutation of tlyA, (2) mutation of one or more 16S or 23S rRNA genes, or (3) mutation of one or more genes for membrane proteins. The tlyA genes of CAP9, CAP15, CAP16 and CAP17, together with those of the control strain M145, were amplified by PCR with oligonucleotides tlyA-F and tlyA-R and the amplified DNA fragments were sequenced with the same oligonucleotides. This showed that the tlyA genes amplified from CAP9, CAP16 and CAP17 all carried a single mutation, which most likely was the cause of the enhanced resistance to capreomycin. The mutations were: insertion of a G residue after nt position 481 in mutant 17, and insertion of a C residue after nt position 585 in mutants CAP9 and CAP15 (see FIG. 23). The sequences are provided in the sequence listing. The tlyA genes amplified from the genomes of S. coelicolor M145 and CAP16 had the wild-type sequence. To establish if methylation of the rRNA was indeed affected in the expected TlyA-dependent manner, construct pGWS405 was introduced in S. coelicolor CAP9 and in its parent M145. pGWS405 is a derivative of the low-copy number shuttle vector pHJI401 (Kieser et al., 2000 carrying wild-type S. coelicolor tlyA expressed from the combined ermE and tuft promoters (promoters were obtained from pGWS4-SD, van Wezel et al. 2000).

By methods described herein, primer extension analysis was used to assess if the 16S and 23S rRNA were methylated at positions C1409 and C1920, respectively. Excitingly, RNA obtained from tlyA mutant CAP9 was not methylated, while rRNA obtained from the same mutant strain complemented with pGWS405 was methylated at both C1409 (16S rRNA) and C1920 (23S rRNA) (FIG. 21). Expectedly, rRNA isolated from the parental strain M145 had both positions methylated. This proves that in the complex actinomycete Streptomyces, which differs from its distant relative Mycobacterium in that they have a complex development and produce antibiotics, TlyA is also fully responsible for methylation of C1409 in 16S rRNA and C1920 in 23S rRNA, and that this effects enhanced sensitivity to capreomycin. Mutation of tlyA causes capreomycin resistance.
Creating an in-Frame Deletion Mutant of S. coelicolor tlyA
To unequivocally prove that the mutations in tlyA were the sole cause of the observed resistance of S. coelicolor, we created an in-frame deletion mutant by removing the entire coding region of tlyA except for the start and stop codons and the final two codons, so as to allow proper transcription and translation of the downstream-located gene (ppnK, SCO1783). For this, we PCR-amplified 1500 bp of the upstream part of tlyA up to and including the ATG start codon and 1500 bp of the downstream region of tlyA, including the last two codons and the stop codon of tlyA, encompassing exactly nt positions −1500/+3 and +808/+2300 relative to the tlyA start codon, respectively. The oligonucleotides were designed such as to add restriction endonuclease sites to the extremities of the amplified DNA fragments, so as to allow the cloning of the upstream part as an EcoRI-BamHI fragment, and the downstream fragment as a BamHI-HindIII fragment, where the BamHI sites were located closest to the gene. The upstream fragment was cloned as an EcoRI-BamHI fragment into pSET151 (Kieser et al. 2000) digested with the same enzymes, followed by insertion of the downstream fragment as a BamHI-HindIII fragment. This yielded construct pGWS410 designed for the in-frame replacement of nt positions 4-807 of the tlyA gene by the sequence GGATCC (for a BamHI site) on the S. coelicolor genome. The construct was introduced in S. coelicolor M145 by transformation, and positive transformants were identified by selecting for thiostrepton resistance. Since pSET151 cannot replicate in streptomycetes (due to lack of a Streptomyces ori), colonies can only become resistant by having the construct stably integrated in the genome. One such colony was selected, replicated onto SFM agar plates without antibiotics and loss of the disruption vector was selected by screening for loss of thiostrepton resistance. 40 such double recombinants were selected and tested for capreomycin resistance. Excitingly, one strain had acquired extremely high capreomycin resistance and grew very well on MM agar plates containing 100 ug/ml capreomycin, which is more than 100-fold higher than the parent M145 (FIG. 22). Analysis of the mutant by PCR showed that indeed the expected deletion had occurred in the mutant. The strain was designated tlyA-IFD. Introduction of pGWS405 expressing wild-type TlyA resulted in regained capreomycin sensitivity, similar to that of the wild-type strain S. coelicolor M145. This underlines that the sole cause of the acquired capreomycin resistance was indeed the lack of an active TlyA protein in the in-frame deletion mutant tlyA-IFD.
In conclusion, TlyA is a capreomycin resistance determinant in antibiotic-producing streptomycetes. On the basis of the identical functions of TlyA in both mycobacteria and streptomycetes, and the proven rRNA 2′ O-methylating activity of TlyA homologues from highly different species such as Geobacillus stearothermophilus, Thermus thermophilus, Serpulina hyodysenteriae, Streptomyces coelicolor, Mycobacterium tuberculosis and Mycobacterium, smegmatis, we claim that removal of TlyA alone is sufficient to significantly enhance capreomycin resistance in all bacteria.

### Experimental Procedures

### Bacterial Strains and Growth Conditions

M. tuberculosis wild-type strains (H37Rv and Beijing D3) and M. smegmatis wild-type strain LR222 were obtained from the culture collection at the Mycobacteriology Laboratory Branch, Centers for Disease Control and Prevention. M. tuberculosis tlyA mutant strains 315-A, C-202 and C-211 and M. smegmatis tlyA mutant strain P2U were isolated as previously described (Maus et al., 2005a). The M. tuberculosis C-401 mutant strain was isolated by the same method as C-202 and C-211 (Maus et al., 2005a) by challenging the wild-type Beijing D3 strain with capreomycin (Sigma) at 10 μg/ml. M. tuberculosis cultures were grown in complete Middlebrook 7119 broth (Remel and Difco) at 37° C. or on Middlebrook 7H10 agar with 10% (v/v) oleic acid-albumin-dextrose-catalase (Difco). M. smegmatis cultures were grown at 37° C. with shaking in Middelbrook 7H9 broth containing 0.2% (v/v) glycerol or on Middlebrook 7H10 agar containing 0.5% (v/v) glycerol.

Escherichia coli strain DH1 was used for cloning and expressing recombinant tlyA, and was grown by standard techniques (Sambrook et al., 1989). The hyperpermeable E. coli strain AS19 (Sekiguchi and Iida, 1967) was additionally used to express tlyA.

Cloning of tlyA
The M. smegmatis LR222 tlyA gene was amplified by PCR using the upstream primer 5′-ccgcatatggcacggcgagctcg and the downstream primer 5′-gctcaaagatctttgcggcccttcctcg. The gene was brought under control of the lac promoter by insertion into the NdeI and BglII sites in the plasmid pLJ102 (a derivative of the expression vector pQE-60 from Qiagen) to form plasmid pSJ101. Both pLJ102 and pSJ101 were used to transform E. coli DH1 and AS19 strains.

### Growth Analyses

M. tuberculosis wild-type Beijing D3 and rrl mutant C-401 were inoculated in triplicate into 7H9 media without drug and 7H9 media containing 10 μg/ml capreomycin, and were grown at 37° C. for 21 days. Growth of the cultures was monitored daily at OD₆₀₀. The MICs of antibiotics were determined for each strain as previously described (Maus et al., 2005a, b).

Overnight cultures of E. coli DH1 and AS19 strains were diluted 10⁵-fold and plated onto agar containing viomycin (US Pharmacopeia), capreomycin, kanamycin or rifampicin (Sigma) with concentrations increasing in two-fold steps. The agar plates were incubated at 37° C.; and after 22 h, MICs were scored as the lowest concentration at which no growth was observed.

Isolation of mycobacterial rRNA
M. tuberculosis wild-type and tlyA mutant bacteria were grown to mid-log phase in 50 ml of 7H9 broth with shaking, and were harvested by centrifugation. Cells were re-suspended in 2 ml Trizol (Invitrogen) and incubated at room temperature for 10 min. RNA was extracted as outlined by DesJardin (DesJardin, 1999), with the exception of the Cleanascite and DNase treatment steps which were omitted.

M. smegmatis strains were grown to late log phase in 200 ml 7H9 broth with shaking, and were harvested by centrifugation. Cells were washed with 10 ml buffer A (10 mM Tris-HCl pH 7.5, 10 mM MgCl₂, 10 mM NH₄Cl, 100 mM KCl) at 4° C., and were lysed by sonication in the same buffer. Cell debris was removed by centrifugation twice at 30,000 g for 10 min. Ribosomes were pelleted from the supernatant by centrifugation at 30,000 g for 18 h at 4° C., and were resuspended in 200 μl cold buffer A. Ribosomal proteins were removed by phenol/chloroform extraction, the rRNA was recovered by ethanol precipitation and was redissolved in H₂O.

### Primer Extension

5′-³²P-end-labelled deoxynucleotide primers were hybridized to complementary regions of rRNA, and were extended with AMV reverse transcriptase (Finnzymes) as described by Stern et al. (Stern et al., 1988). Initially, primers complementary to the following seven regions were used: 1461-1510, 1505-1524, and 1529-1543 in 16S rRNA; and 803-821, 1955-1974, 2115-2131, and 2591-2610 in 23S rRNA (E. coli numbering). Sites of 2′-O-ribose methylation were detected by reducing dNTP concentrations (Maden et al., 1995). After the initial screening, 16S rRNA nucleotide C1409 was analyzed more closely from a 1412-1432 primer using decreasing concentrations of dGTP (40 μM, 0.2 μM, 0.1 μM and 0.04 μM) while maintaining dATP, dCTP and ddTTP at 40 μM. Nucleotide C1920 in 23S rRNA was analyzed in a similar manner from a primer complementary to nucleotides 1925-1942. Extension products were run on denaturing polyacrylamide/urea gels alongside dideoxy sequencing reactions performed on M. smegmatis P2U rRNAs. Gels bands were visualized using a Typhoon Phosphorimager (Amersham Biosciences).

### MALDI-TOF Mass Spectrometry Analysis

Specific stretches of rRNA approximately 50 nucleotides in length (FIG. 1) were isolated and purified for mass spectrometry analysis by hybridization to oligodeoxynucleotides (Andersen et al., 2004). Mycobacterial rRNA at 30 to 100 pmol was heated with a ten-fold molar excess of oligodeoxynucleotide for 1 min. at 80 to 90° C., and cooled slowly over 2 h to 45° C. The DNA-rRNA hybrids were digested with nucleases, and the protected rRNA fragments were isolated and purified (Andersen et al., 2004). RNA fragments (2.5 pmol in 1 μl H₂O) were digested with either 0.25 μg RNase A or 10 units of RNase T1 (USB) in 0.5 μl 0.5 M 3-hydroxypicolinic acid (3-HPA) at 37° C. for 2 h. Cyclic phosphates were hydrolyzed with HCl; the RNA oligonucleotides were dried and redissolved in H₂O.

Mass spectra were recorded in reflector and positive ion mode on a PerSeptive Voyager-DE STR mass spectrometer (Applied Biosystems); spectra were smoothed using the software “m/z” (Proteometrics Inc). Tandem mass spectra were recorded in positive ion mode on a MicroMass MALDI Q-TOF Ultima mass spectrometer (Kirpekar and Krogh, 2001). The collision energy used for tandem mass spectrometry was varied between 30 and 110 eV. All tandem mass spectra were smoothed using the MassLynx software supplied by the manufacturer.

Isolation and Cloning of tlyA Genes
The tlyA genes were amplified from genomic DNA that had been extracted from the respective bacteria. For each tlyA gene, two sets of PCR primers were used. First, the DNA sequence including the tlyA gene plus about 30 nucleotides upstream and downstream were amplified using primers that are perfectly complementary to these regions of the genomic DNA (e.g. for M. smegmatis DNA using primers SJ10 and SJ11 in Table 3). This amplification product was then subjected to nested PCR using the second set of primers (e.g. for M. smegmatis DNA using primers SJ8 and SJ9 in Table 3). This amplified the sequence corresponding exactly to the coding sequence of the tlyA gene (defined for each species in Table 2), and placed an Nde1 site at the ATG start codon and a BglII site (or in some cases a BamHI site) at the end of the coding sequence. The nested PCR products were cloned into the same sites in the plasmid expression vector pLJ102 (a derivative of the plasmid pQE-60 from Qiagen). Final constructs contain the tlyA sequence immediately after the lac promoter, and express TlyA with six histidines at the C-terminal end. The plasmid has a β-lactamase gene (Amp^{R}).

### Method to Determine Drug Binding to Both Ribosomal Subunits Across Ribosomal Interface

Ribosomal particles were isolated from Mycobacterium smegmatis. The particles were kept as associated ribosomes (70S), or were dissociated and purified as ribosomal subunits (30S and 50S). The cyclic peptide antibiotics capreomycin and viomycin were added to 30S, 50S and 70S particles at a molar excess of drug to ribosomal particles. The interactions of the drugs were footprinted using the reagents dimethyl sulphate (DMS) and hydroxyl radical (OH*). Oligodeoxynucleotide primers that were used for the reverse transcriptase analyses of the DMS and OH* modification sites are given in Table 1. The differences in modification patterns (footprints) reveal the interaction sites of the drugs plus any structural rearrangements that occur within or between the ribosomal subunits.

### REFERENCES

Andersen, T. E., Porse, B. T., and Kirpekar, F. (2004) A novel partial modification at C2501 in Escherichia coli 23S ribosomal RNA. Rna 10: 907-913.
Brimacorabe, R. P., Mitchel, P., Oswald, M., Stade, K., and Bochkariov, D. (1993) Clustering of modified nucleotides at the functional center of bacterial ribosomal RNA. FASEB Journal 7: 161-167.
Cannon, J. J., Subramanian, S., Schnare, M. N., Collett, J. R., D'Souza, L. M., Du, Y., Feng, B., Lin, N., Madabusi, L. V., uuml, Her, K. M., Pande, N., Shang, Z., Yu, N., and Gutell, R. R. (2002) The Comparative RNA Web (CRW) Site: an online database of comparative sequence and structure information for ribosomal, intron, and other RNAs. BMC Bioinformatics 3: 2-32.
Caldas, T., Binet, E., Bouloc, P., Costa, A., Desgres, J., and Richarme, G. (2000) The FtsJ/RrmJ heat shock protein of Escherichia coli is a 23 S ribosomal RNA methyltransferase. J Biol Chem 275: 16414-16419.
Cundliffe, E. (1989) How antibiotic-producing organisms avoid suicide. Annu Rev Microbiol 43: 207-233.
del Campo, M., Ofengand, J., Malhotra, A. (2004) Crystal structure of the catalytic domain of RluD, the only rRNA pseudouridine synthase required for normal growth of Escherichia coli. RNA 10: 231-239.
DesJardin, L. E. (1999) Isolation of Mycobacterium tuberculosis RNA from sputum. Methods Mol. Med: 133-139.
Douthwaite, S., Fourmy, D., and Yoshizawa, S. (2005) Nucleotide methylations in rRNA that confer resistance to ribosome-targeting antibiotics. In Fine-tuning of RNA functions by modification and editing. Vol. 12. Grosjean, H. (ed). New York: Springer Verlag, pp. 287-309.
Dye, C., Espinal, M. A., Watt, C. J., Mbiaga, C., and Williams, B. G. (2002) Worldwide incidence of multidrug-resistant tuberculosis. J Infect Dis 185: 1197-1202.
Gale, E. F., Cundliffe, E., Reynolds, P. E., Richmond, M. H., and Waring, M. J. (1981) The Molecular Basis of Antibiotic Action. London: John Wiley and Sons.
Gastaldo, L., and Marinelli, F. (2003) Changes in GE2270 antibiotic production in Planobispora rosea through modulation of methylation metabolism. Microbiol. 149: 1523-32.
Gustafsson, C., and Persson, B. C. (1998) Identification of the rrmA gene encoding the 23S rRNA m1G745 methyltransferase in Escherichia coli and characterization of an m1G745-deficient mutant. J Bacteriol 180: 359-365.
Gutell, R. R., Larsen, N., and Woese, C. R. (1994) Lessons from an evolving rRNA: 16S and 23S rRNA structures from a comparative perspective. Microbiological Reviews 58: 10-26.
Helser, T. L., Davies, J. E., and Dahlberg, J. E. (1972) Mechanism of kasugamycin resistance in Escherichia coli. Nat New Biol 235: 6-9.
Joseph, S., Weiser, B., and Noller, H. F. (1997) Mapping the inside of the ribosome with an RNA helical ruler. Science 278: 1093-1098.
Kirpekar, F., and Krogh, T. N. (2001) RNA fragmentation studied in a matrix-assisted laser desorption/ionisation tandem quadrupole/orthogonal time-of-flight mass spectrometer. Rapid Commun Mass Spectrom 15: 8-14.
Liou, Y. F., and Tanaka, N. (1976) Dual actions of viomycin on the ribosomal functions. Biochem Biophys Res Commun 71: 477-483.
Liu, M., and Douthwaite, S. (2002a) Methylation at nucleotide G745 or G748 in 23S rRNA distinguishes Gram-negative from Gram-positive bacteria. Mol Microbiol 44: 195-204.
Liu, M., and Douthwaite, S. (2002b) Activity of the ketolide telithromycin is refractory to Erm monomethylation of bacterial rRNA. Antimicrob Agents Chemother 46: 1629-1633.
Maden, B. E., Corbett, M. E., Heeney, P. A., Pugh, K., and Ajuh, P. M. (1995) Classical and novel approaches to the detection and localization of the numerous modified nucleotides in eukaryotic ribosomal RNA. Biochimie 77: 22-29.
Maher, D., and Raviglione, M. (2005) Global epidemiology of tuberculosis. Clin Chest Med 26: 167-182, v.
Maivali, U., and Remme, J. (2004) Definition of bases in 23S rRNA essential for ribosomal subunit association. Rna 10: 600-604.
Maus, C. E., Plikaytis, B. B., and Shinnick, T. M. (2005a) Mutation of tlyA confers capreomycin resistance in Mycobacterium tuberculosis. Antimicrob Agents Chemother 49: 571-577.
Maus, C. E., Plikaytis, B. B., and Shinnick, T. M. (2005b) Molecular analysis of cross-resistance to capreomycin, kanamycin, amikacin, and viomycin in Mycobacterium tuberculosis. Antimicrob Agents Chemother 49: 3192-3197.
McLuckey, S. A., Van Berkel, G. J., and Glish, G. L. (1992) Tandem mass spectrometry of small multiply charges oligonucleotides. J Am Soc Mass Spectrom 3: 60-70.
Merryman, C., Moazed, D., Daubresse, G., and Noller, H. F. (1999a) Nucleotides in 23S rRNA protected by the association of 30S and 50S ribosomal subunits. J Mol Biol 285: 107-113.
Merryman, C., Moazed, D., McWhirter, J., and Noller, H. F. (1999b) Nucleotides in 16S rRNA protected by the association of 30S and 50S ribosomal subunits. J Mol Biol 285: 97-105.
Misumi, M., Tanaka, N., and Shiba, T. (1978) Binding of ¹⁴C-tuberactinomycin O, an antibiotic closely related to viomycin, to the bacterial ribosome. Biochem Biophys Res Common 82: 971-976.
Mitchell, P., Osswald, M., and Brimacombe, R. (1992) Identification of intermolecular RNA cross-links at the subunit interface of the Escherichia coli ribosome. Biochemistry 31: 3004-3011.
Moazed, D., and Noller, H. F. (1987) Chloramphenicol, erythromycin, carbomycin and vernamycin B protect overlapping sites in the peptidyl transferase region of 23S ribosomal RNA. Biochimie 69: 879-884.
Moazed, D., and Noller, H. F. (1989a) Interaction of tRNA with 23S rRNA in the ribosomal A, P, and E sites. Cell 57: 585-597.
Moazed, D., and Noller, H. F. (1989b) Intermediate states in the movement of transfer RNA in the ribosome. Nature 342: 142-148.
Moazed, D., and Noller, H. F. (1990) Binding of tRNA to the ribosomal A and P sites protects two distinct sets of nucleotides in 16 S rRNA. J Mol Biol 211: 135-145.
Modolell, J., and Vazquez (1977) The inhibition of ribosomal translocation by viomycin. Eur J Biochem 81: 491-497.
Noller, H. F. (1984) Structure of ribosomal RNA. Annual Review of Biochemistry 53: 119-162.
Poehlsgaard, J., and Douthwaite, S. (2005) The bacterial ribosome as a target for antibiotics. Nature Reviews Microbiology 3: 870-881.
Rausch C., Weber T., Kohlbacher O., Wohlleben W., and Huson D. H. (2005) Specificity prediction of adenylation domains in nonribosomal peptide synthetases (NRPS) using transductive support vector machines (TSVMs). Nucleic Acids Res. 33: 5799-5833.
Rozenski, J., Crain, P. F., and McCloskey, J. A. (1999) The RNA Modification Database: 1999 update. Nucl Acids Res 27: 196-197.
Sambrook, J., Fritsch, E. F., and Maniatis, T. (1989) Molecular Cloning: A Laboratory Manual. Cold Spring Harbor, N.Y.: Cold Spring Harbor Press.
Schlunzen, F., Zarivach, R., Harms, J., Bashan, A., Tocilj, A., Albrecht, R., Yonath, A., and Franceschi, F. (2001) Structural basis for the interaction of antibiotics with the peptidyl transferase centre in eubacteria. Nature 413: 814-821.
Schuwirth, B. S., Borovinskaya, M. A., Hau, C. W., Zhang, W., Vila-Sanjurjo, A., Holton, J. M., and Cate, J. H. (2005) Structures of the bacterial ribosome at 3.5 A resolution. Science 310: 827-834.
Sekiguchi, M., and Iida, S. (1967) Mutants of Escherichia coli permeable to actinomycin. Proc. Natl. Acad. Sci. USA. 58: 2315-2320.
Sharma, S. K., and Mohan, A. (2004) Multidrug-resistant tuberculosis. Indian J Med Res 120: 354-376.
Skinner, R. H., and Cundliffe, E. (1980) Resistance to the antibiotics viomycin and capreomycin in the Streptomyces species which produce them. J. Gen. Microbiol. 120: 95-104.
Stern, S., Moazed, D., and Noller, H. F. (1988) Structural analysis of RNA using chemical and enzymatic probing monitored by primer extension. Methods Enzymol 164: 481-489.
Suzuki, Y., Katsukawa, C., Tamaru, A., Abe, C., Makino, M., Mizuguchi, Y., and Taniguchi, H. (1998) Detection of kanamycin-resistant Mycobacterium tuberculosis by identifying mutations in the 16S rRNA gene. J Clin Microbiol 36: 1220-1225.
Taniguchi, H., Chang, B., Abe, C., Nikaido, Y., Mizuguchi, Y., and Yoshida, S. I. (1997) Molecular analysis of kanamycin and viomycin resistance in Mycobacterium smegmatis by use of the conjugation system. J Bacteriol 179: 4795-4801.
Thomas, M. G., Chan, Y. A., and Ozanick, S. G. (2003) Deciphering tuberactinomycin biosynthesis: isolation, sequencing, and annotation of the viomycin biosynthetic gene cluster. Antimicrob Agents Chemother 47: 2823-2830.
Tu, D., Blaha, G., Moore, P. B., and Steitz, T. A. (2005) Structures of MLS_{B}K antibiotics bound to mutated large ribosomal subunits provide a structural explanation for resistance. Cell 121: 257-270.
Valle, M., Zavialov, A., Li, W., Stagg, S. M., Sengupta, J., Nielsen, R. C., Nissen, P., Harvey, S. C., Ehrenberg, M., and Frank, J. (2003a) Incorporation of aminoacyl-tRNA into the ribosome as seen by cryo-electron microscopy. Nat Struct Biol 10: 899-906.
Valle, M., Zavialov, A., Sengupta, J., Rawat, U., Ehrenberg, M., and Frank, J. (2003b) Locking and unlocking of ribosomal motions. Cell 114: 123-134.
Van Buul, C. P., Damm, J. B., and Van Knippenberg, P. H. (1983) Kasugamycin resistant mutants of Bacillus stearothermophilus lacking the enzyme for the methylation of two adjacent adenosines in 16S ribosomal RNA. Mol Gen Genet 189: 475-478.
Vázquez, D. (1979) Inhibitors of Protein Biosynthesis. Berlin: Springer-Verlag.
Yamada, T., Mizugichi, Y., Nierhaus, K. H., and Wittmann, H. G. (1978) Resistance to viomycin conferred by RNA of either ribosomal subunit. Nature 275: 460-461.
Yamada, T., and Nierhaus, K. H. (1978) Viomycin favours the formation of 70S ribosome couples. Mol Gen Genet 161: 261-265.
Yusupov, M. M., Yusupova, G. Z., Baucom, A., Lieberman, K., Earnest, T. N., Cate, J. H., and Noller, H. F. (2001) Crystal structure of the ribosome at 5.5 Å resolution. Science 292: 883-896.

### BRIEF DESCRIPTION OF THE DRAWINGS

FIG. 1.

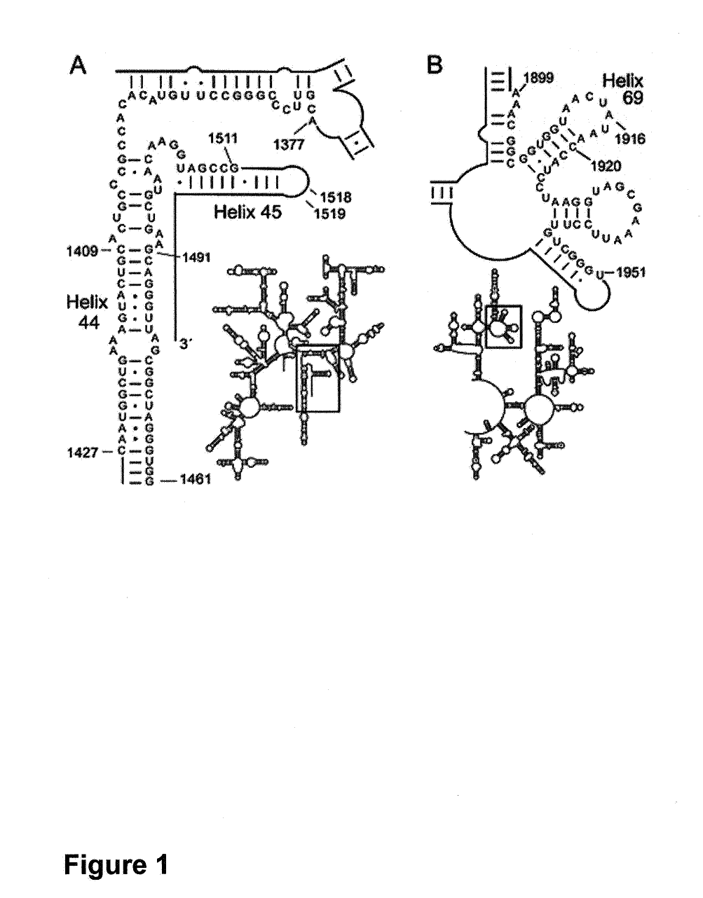

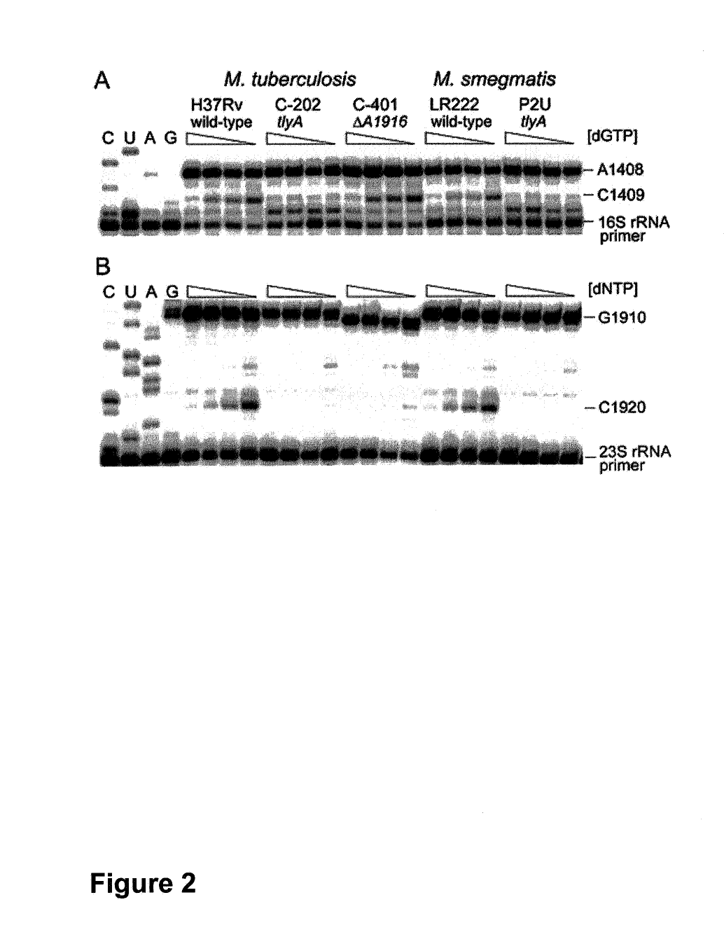

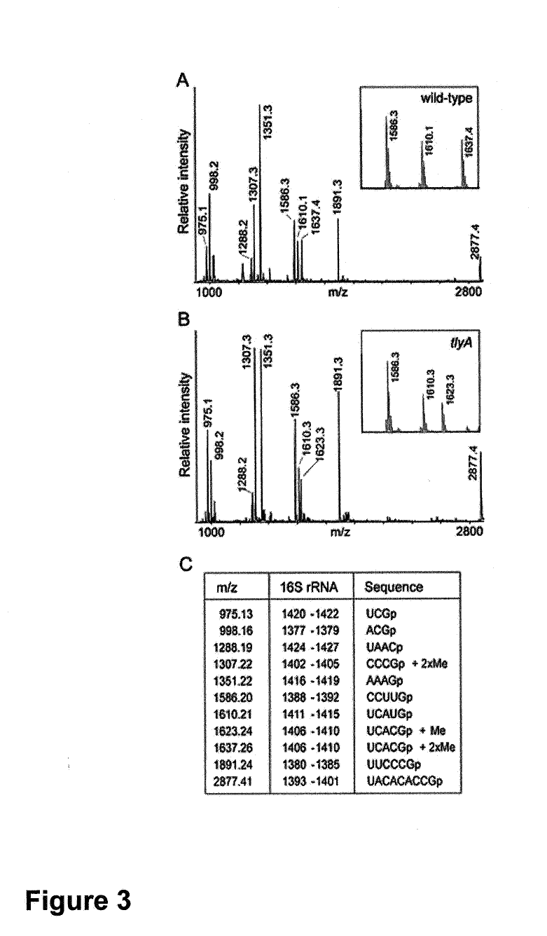

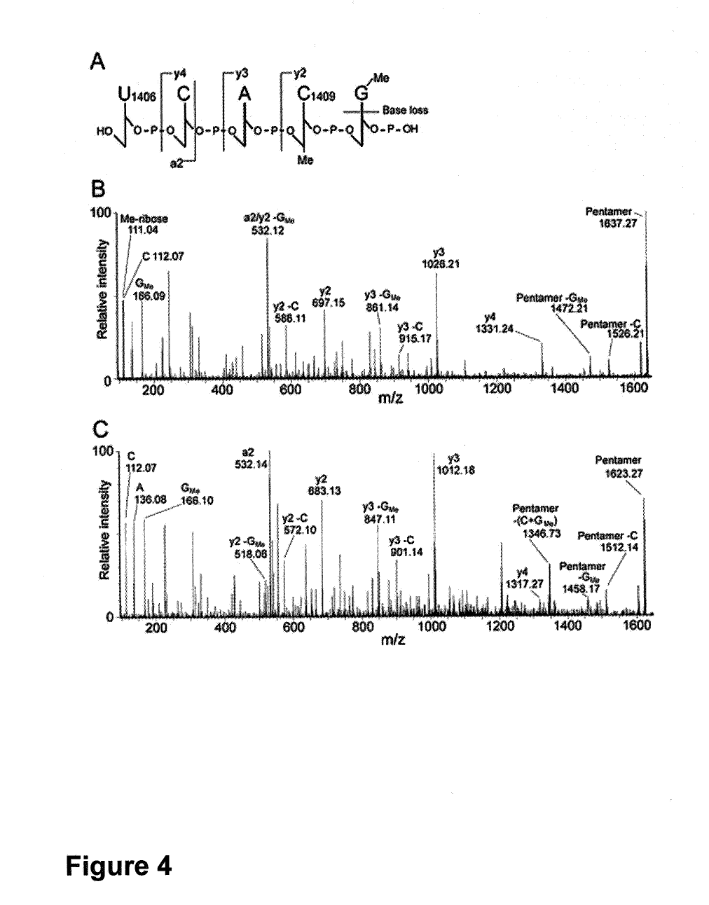

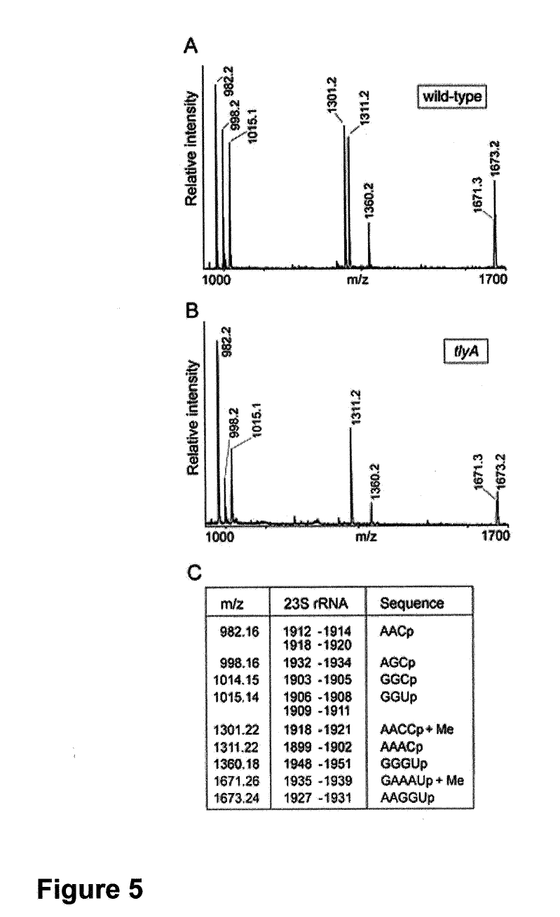

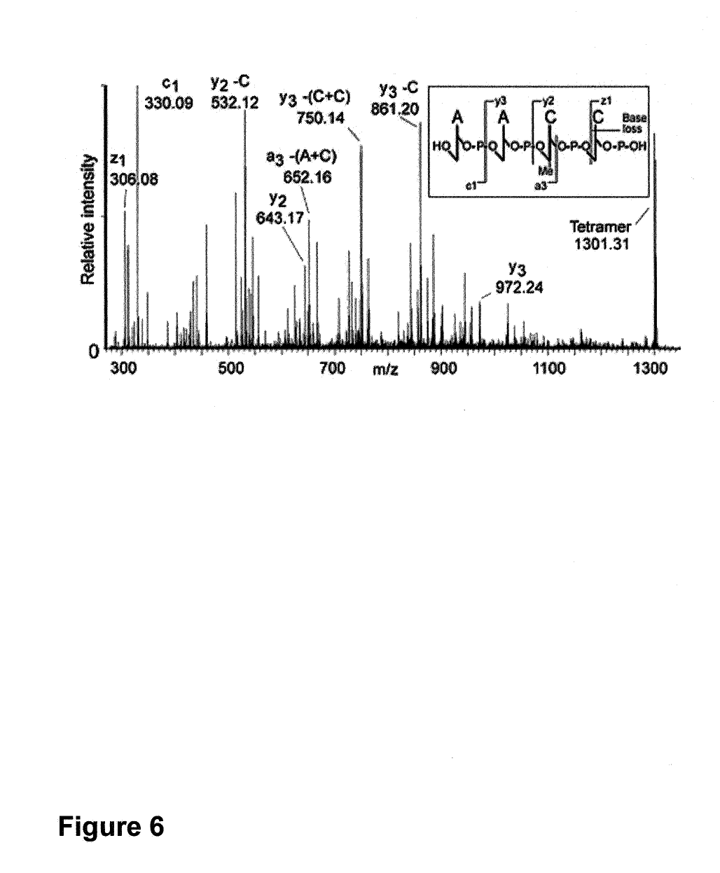

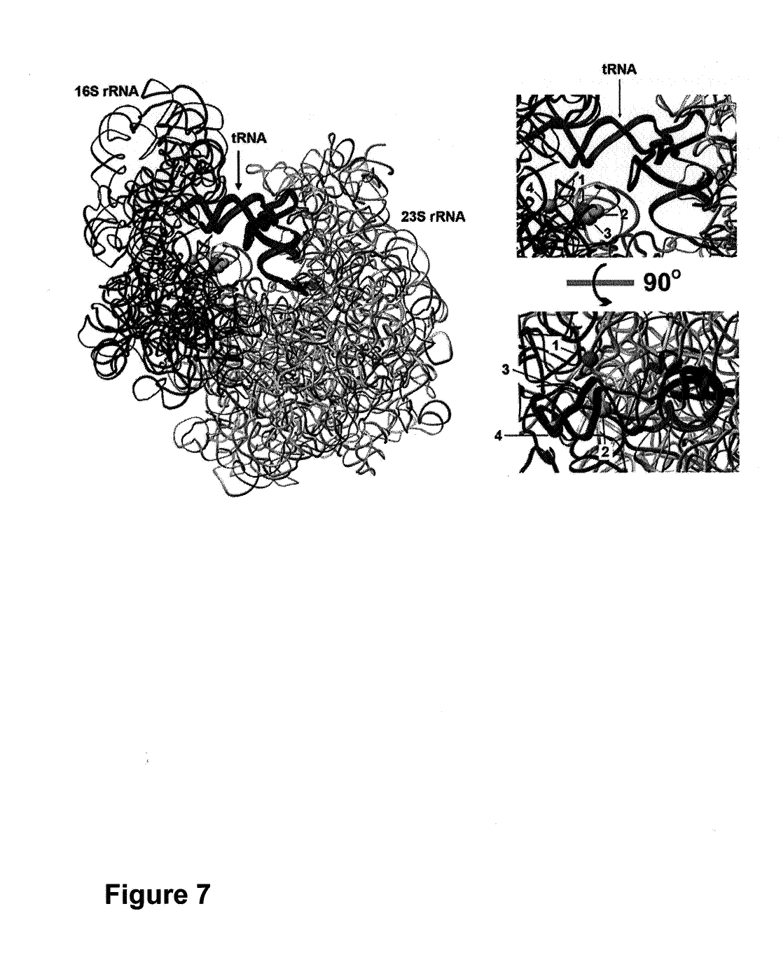

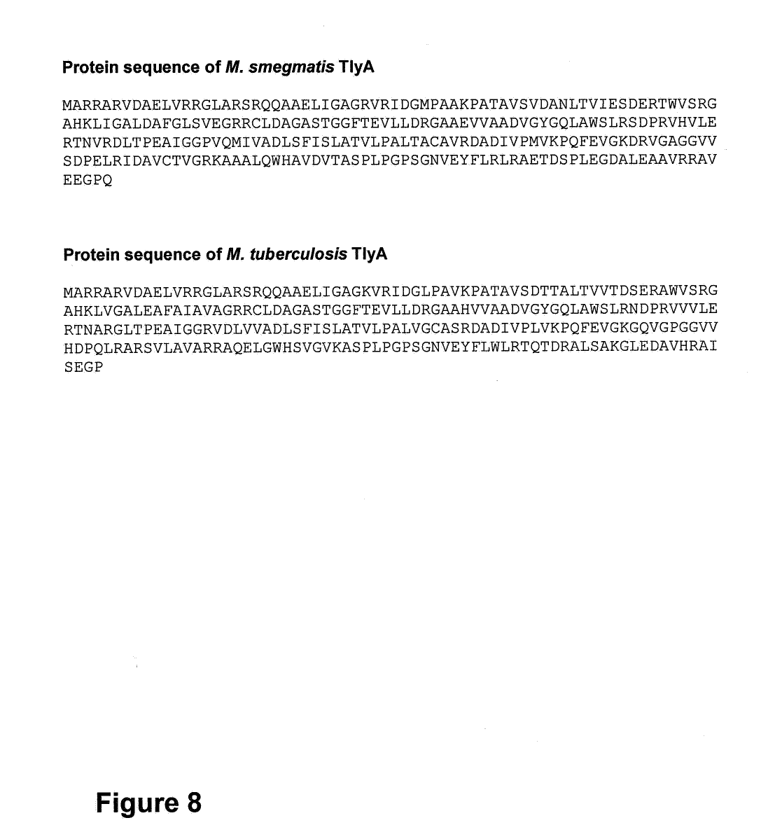

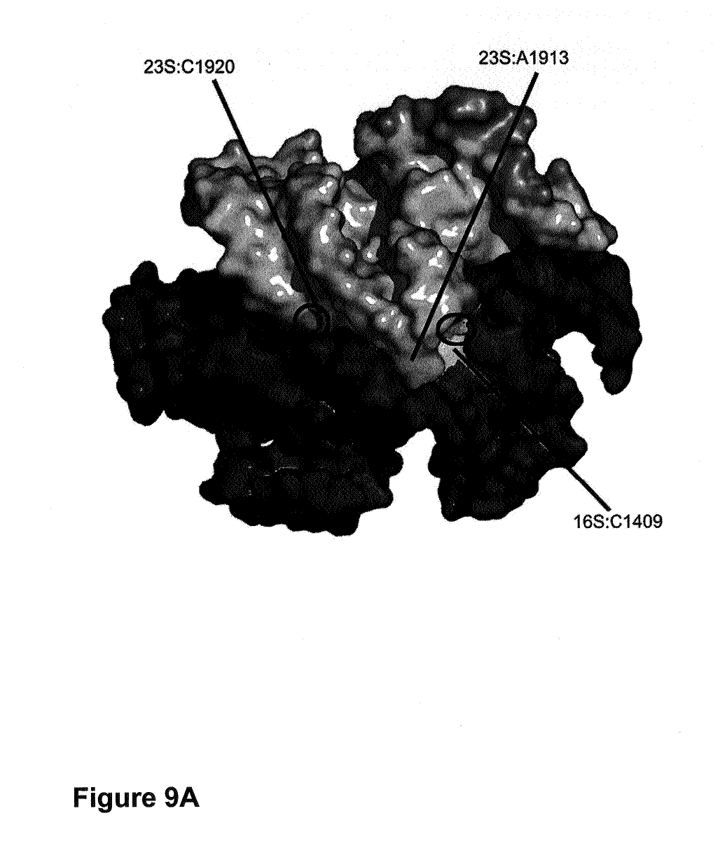

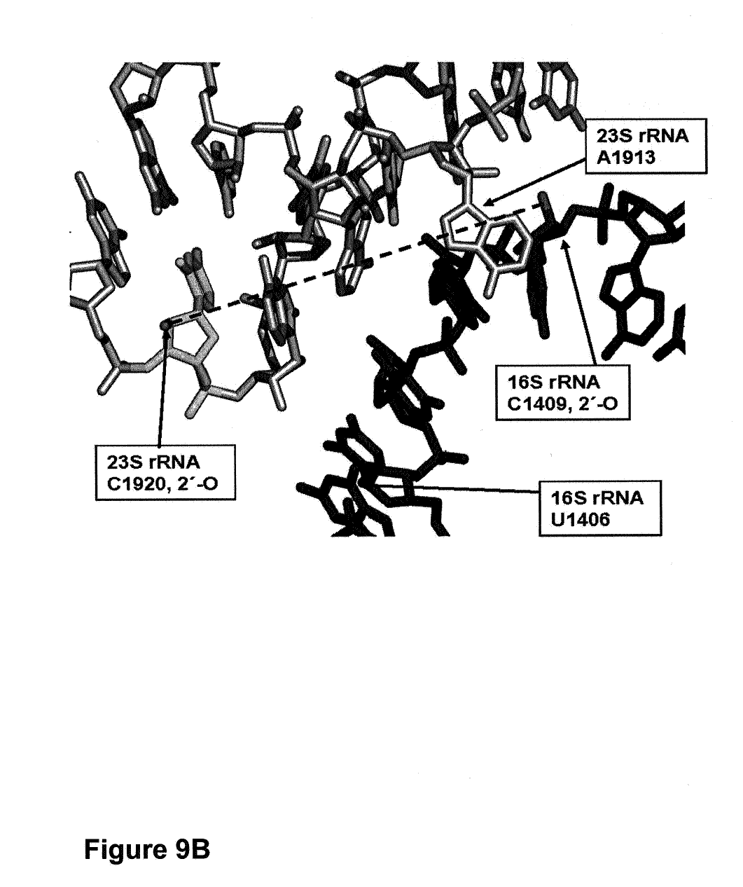

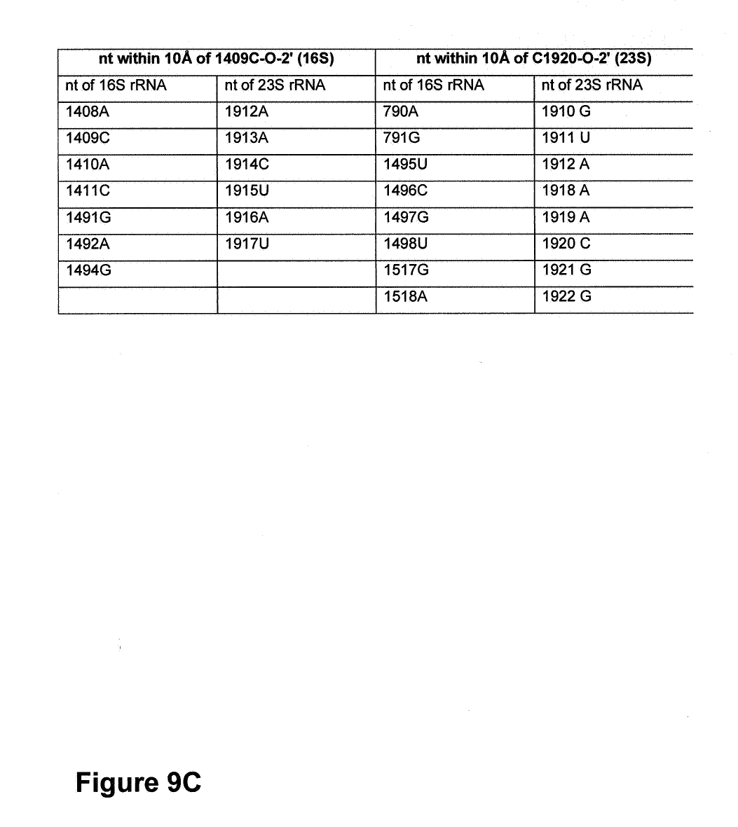

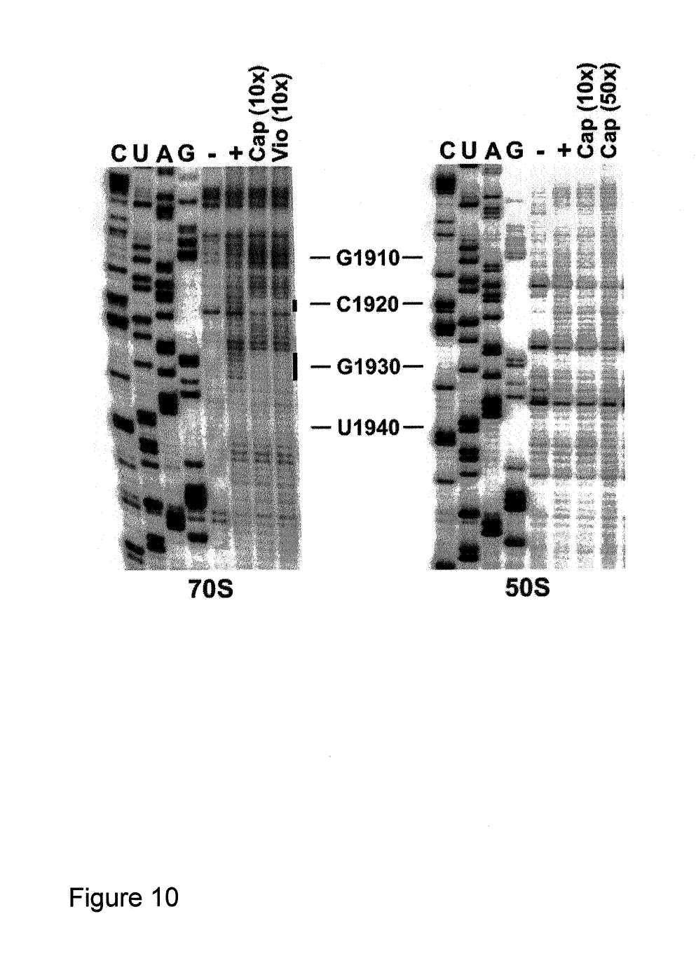

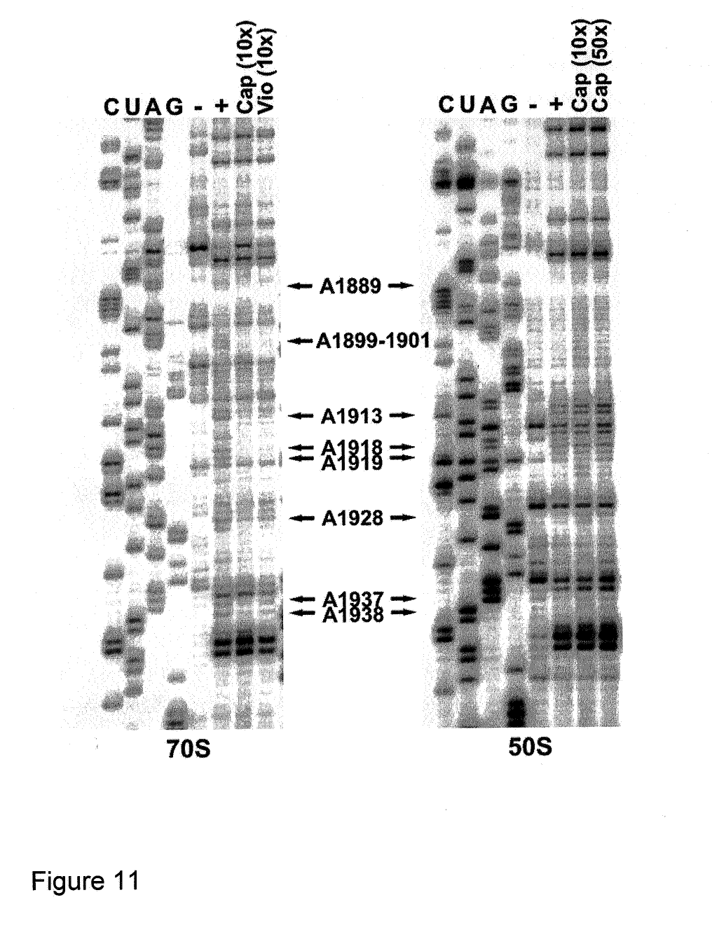

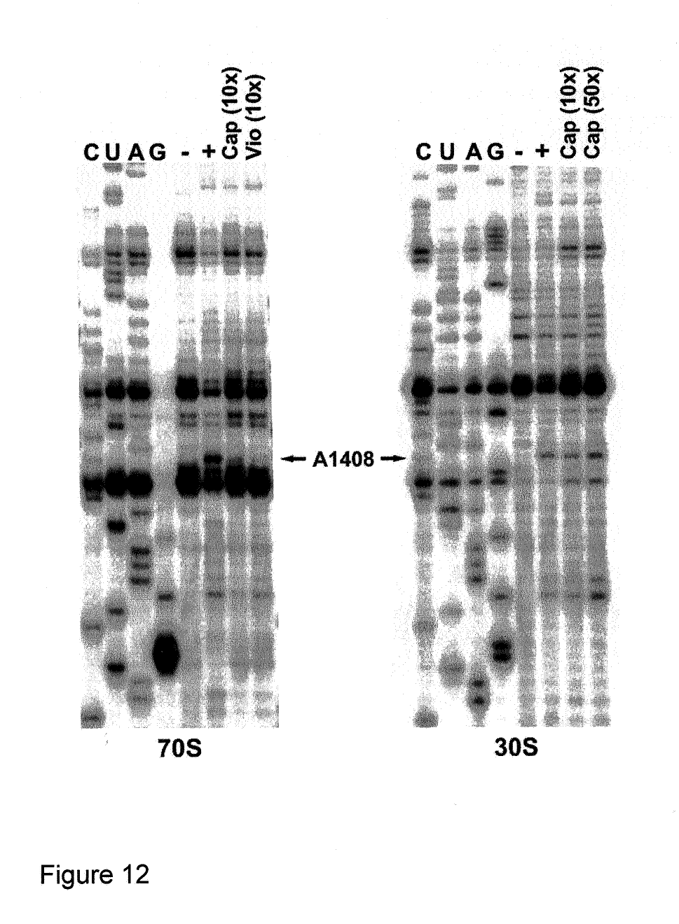

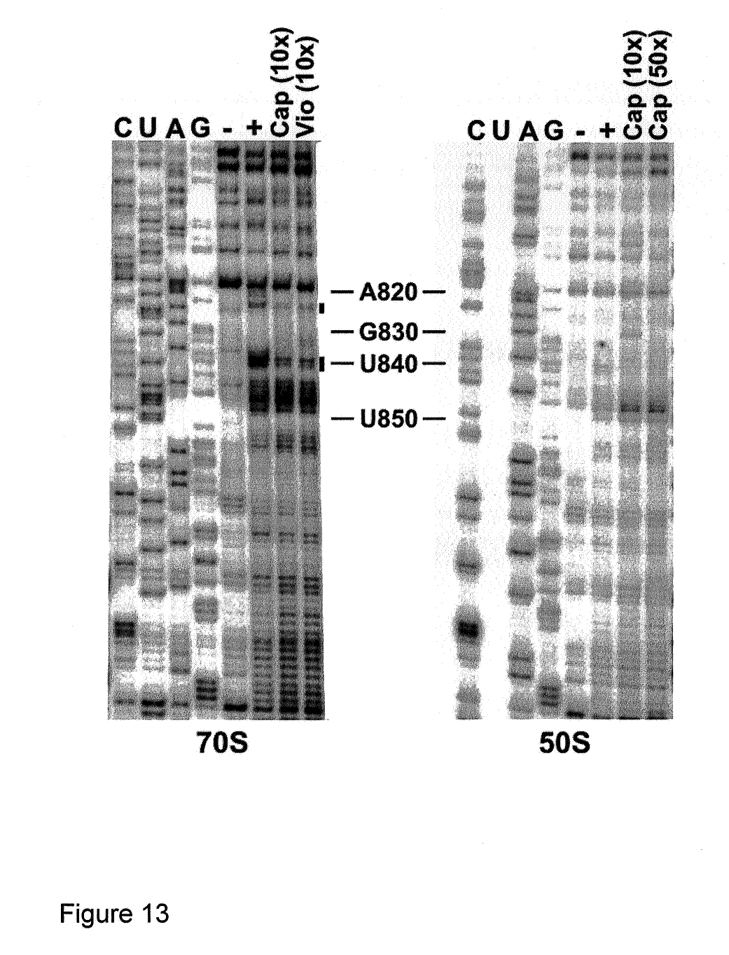

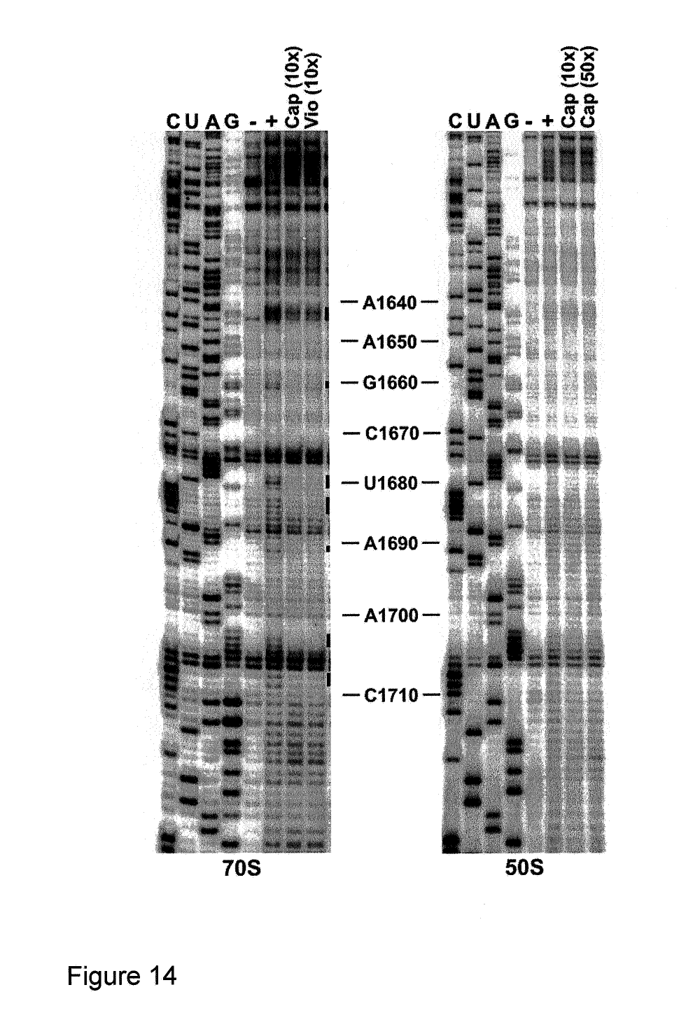

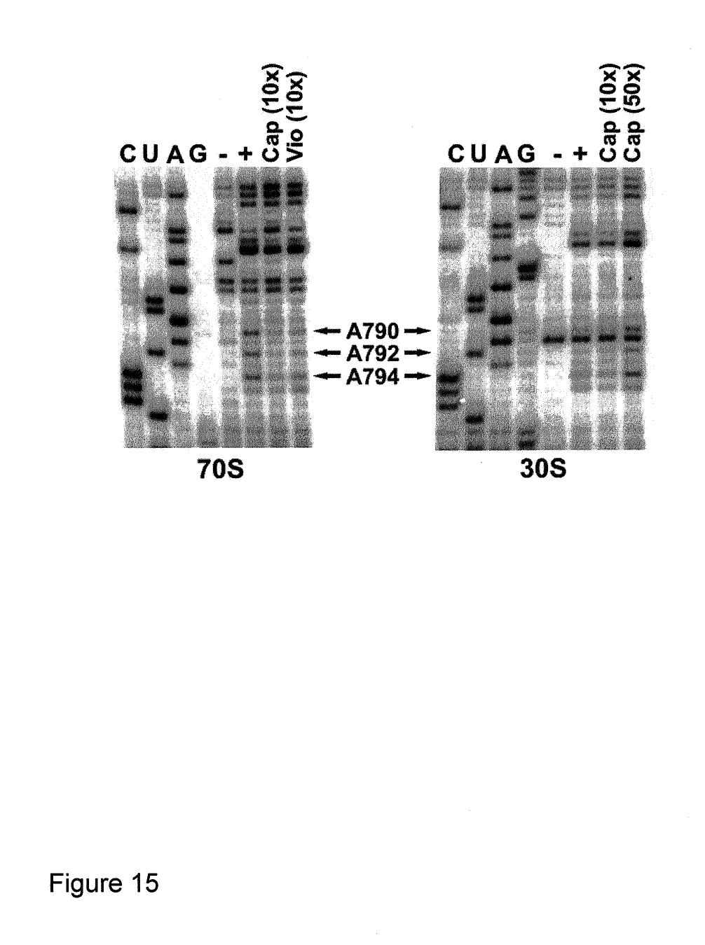

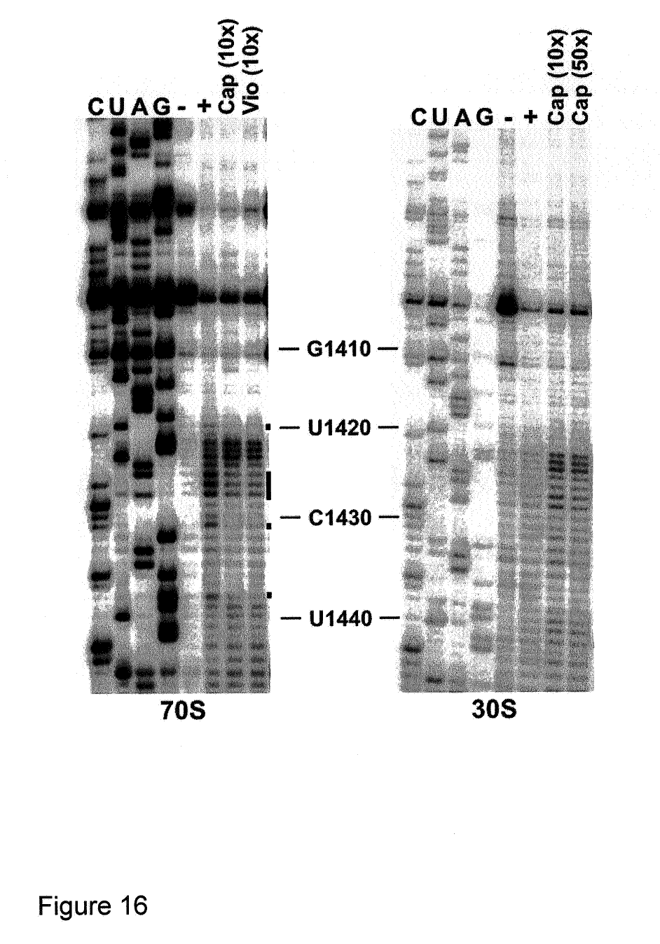

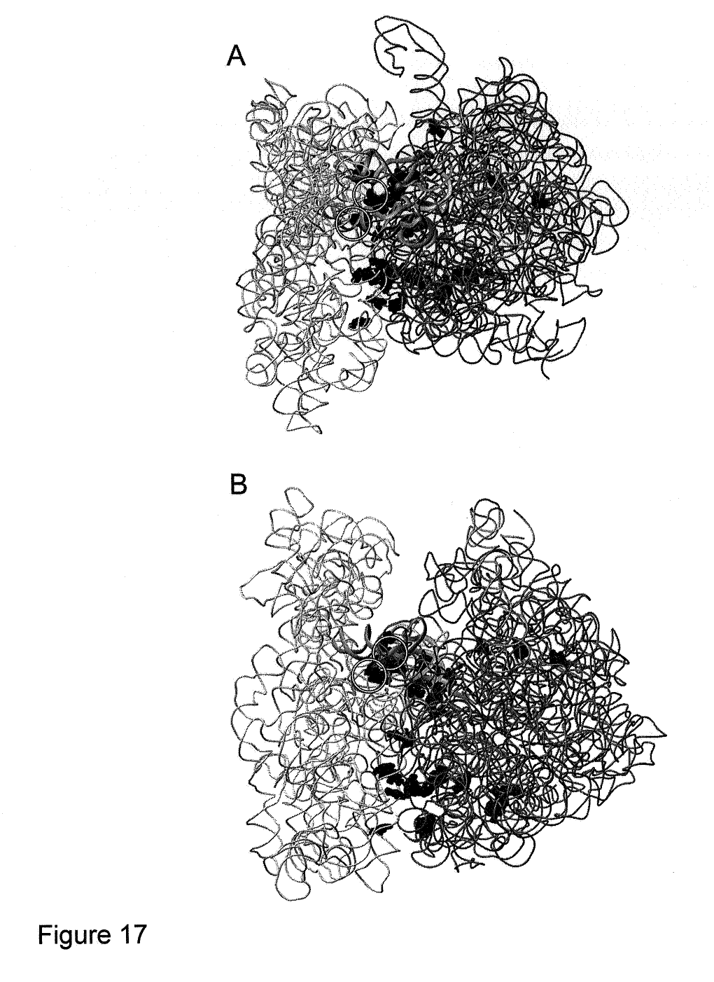

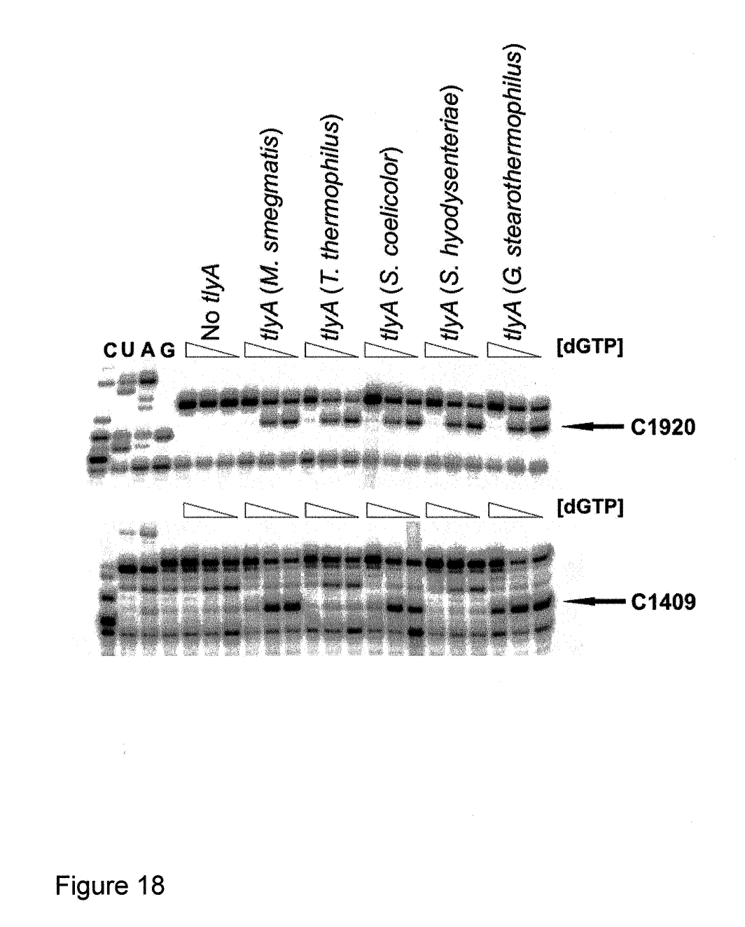

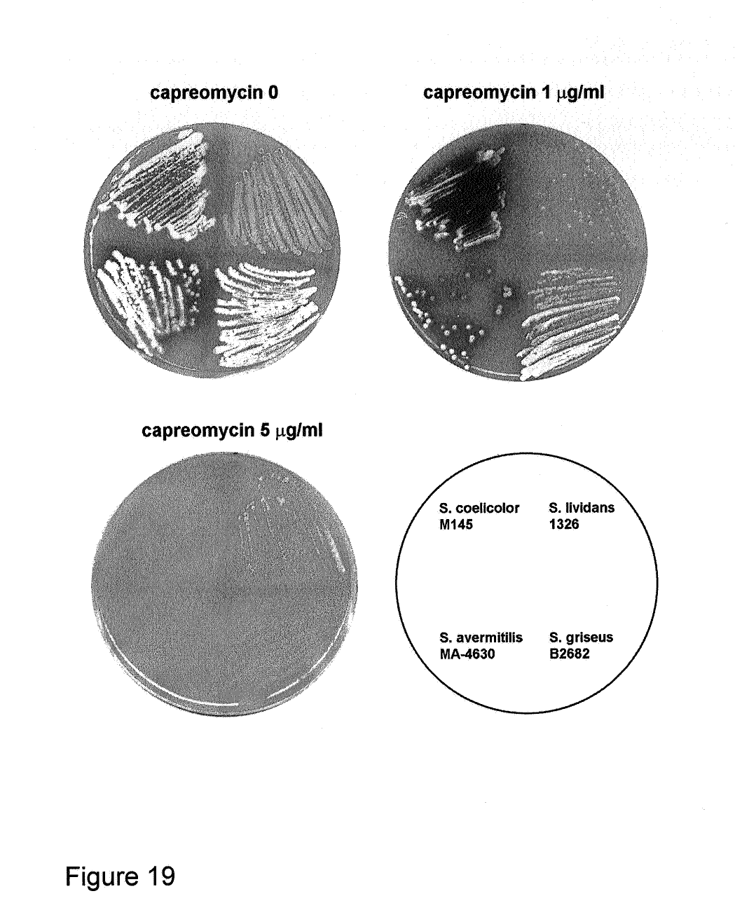

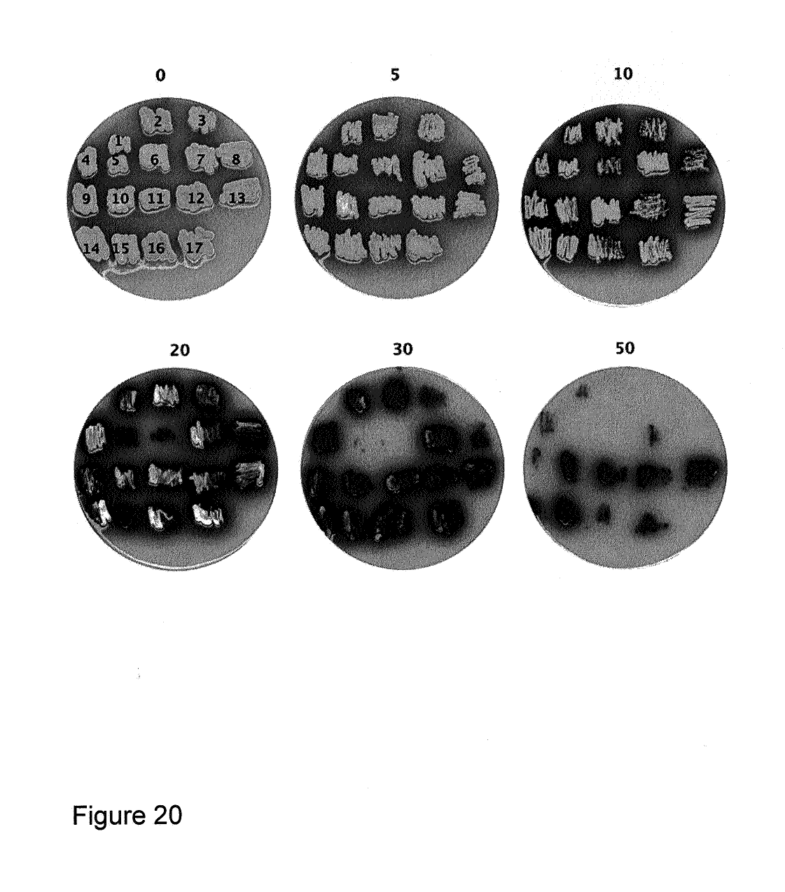

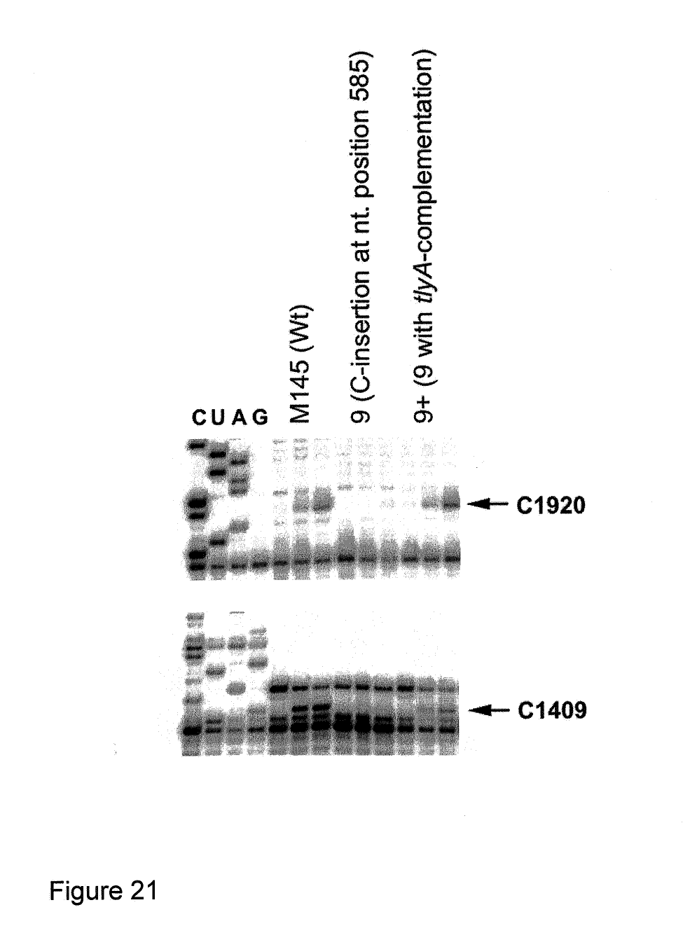

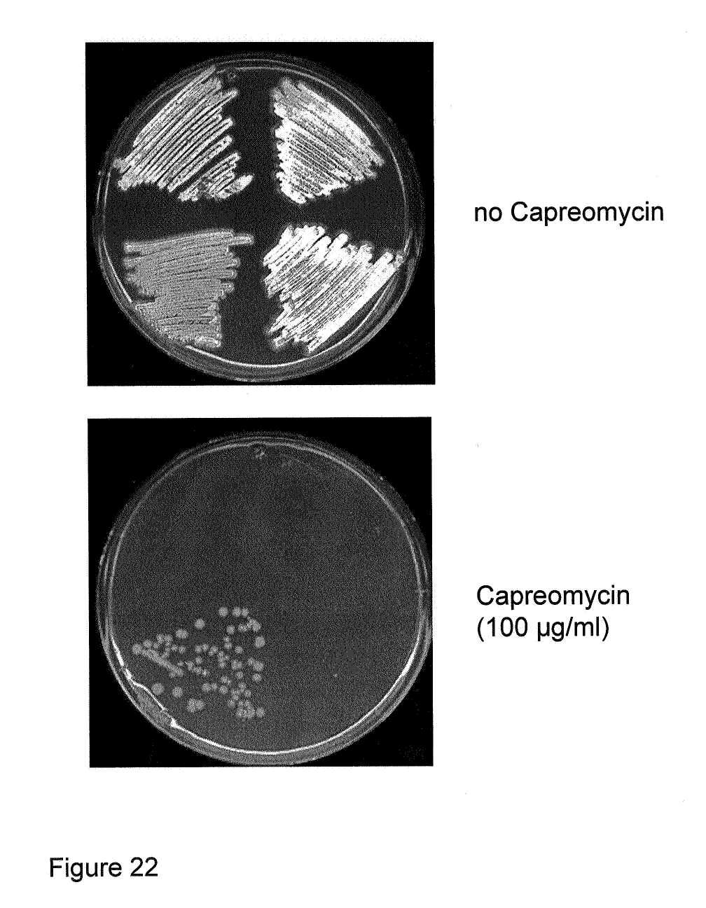

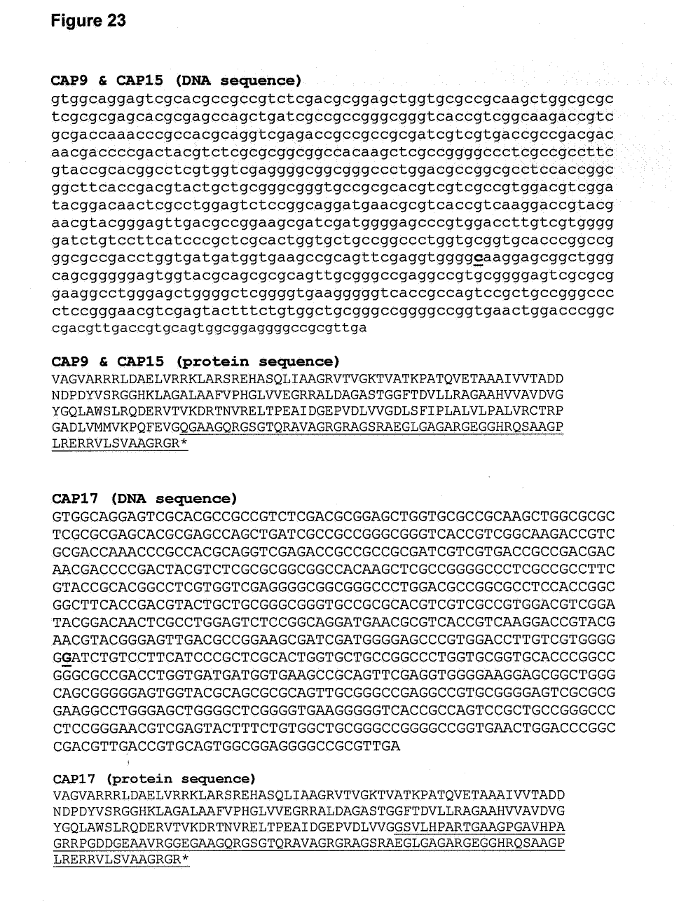

Secondary structures of (A) the 16S rRNA and (B) the 3′-half of 23S rRNA. Sequences isolated by oligodeoxynucleotide hybridization for MS analysis are shown for (A) the A1377-C1427 and G1461-G1511 regions of Mycobacterium 16S rRNA, and (B) the A1899-U1951 region of Mycobacterium 23S rRNA. The E. coli numbering system for rRNA nucleotides (Cannone et al., 2002; Noller, 1984) is used throughout. For reference, the 16S rRNA nucleotide C1409 corresponds to C1402 in M. tuberculosis and C1392 in M. smegmatis; the 23S rRNA nucleotide C1920 corresponds to C2158 in M. tuberculosis and C2144 in M. smegmatis.

FIG. 2.

Gel autoradiographs of primer extension on the rRNAs from wild-type and mutant mycobacterial strains. Decreasing the dNTP concentration (indicated by wedges) intensifies reverse transcription termination at nucleotides with 2′-O-methylation, as is shown here for the wild-type strains (H37Rv and LR222) at (A) C1409 in 16S rRNA, and (B) C1920 in 23S rRNA. No termination occurs here in the tlyA mutant rRNAs (C-202 and C-211); the 23S rRNA A1916-deletion mutant (C-401) shows no C1920 stop, but has retained the C1409 methylation stop. Reverse transcription was terminated completely at nucleotides A1408 and G1910 by inclusion of ddTTP and ddCTP in the respective extensions; the nucleotide deletion in C-401 is apparent in the shift of the G1910 band. M. smegmatis P2U rRNA was used as the template for the dideoxy-sequencing reactions (lanes C, U, A and G).

FIG. 3.

MALDI-TOF mass spectra of RNase T1 oligonucleotides from the mycobacterial 16S rRNA C1409 region. (A) Wild-type M. smegmatis LR222 rRNA shows a peak at m/z 1637 corresponding to UCACGp (U1406-G1410) with two methyl groups. Identical spectra were obtained for the M. tuberculosis wild-type rRNAs (Beijing D3 and H37Rv) and for the A1916-deletion mutant, C-401 (not shown). (B) The UCACGp oligo from the M. smegmatis tlyA mutant P2U runs at m/z 1623, and thus contains only one methyl group; identical spectra were obtained for the M. tuberculosis tlyA mutants C-202, C-211 and 315A (not shown). (C) The theoretical masses of protonated RNase T1 fragments from the A1377-C1427 sequence match the empirically measured m/z values to within 0.1 Da in all cases.

FIG. 4.

Tandem mass spectra of the 16S rRNA sequence U1406-G1410.

(A) The M. tuberculosis H37Rv wild-type oligo (theoretical m/z of 1637.26, measured here at m/z 1637.20) was fragmented resulting in a series of a, b, c and d ions from the 5′-end, and w, x, y and z ions from the 3′-end (McLuckey et al., 1992); for clarity, only a few significant ions are shown. One methyl group, on nucleotide G1410, is evident from the mass of the x1 ion (m/z 440.10), and was localized to the guanine base, which is lost in the y2 ion (m/z 532.10) and appears at m/z 166.07. The second methyl group is at C1409 and could be seen in (for example) the y2 ion (m/z 697.15). This methyl group is retained on the y2 ion at m/z 586.11 despite loss of the cytosine nucleobase; the methyl group is thus on the C1409 ribose, which can be seen at m/z 111.04. The same methylation pattern was seen for the wild-type strains, M. tuberculosis Beijing D3 and M. smegmatis LR222, as well as for the A1916-deletion mutant C-401.

(B) The corresponding spectrum from the C-211 tlyA mutant (theoretical oligo m/z 1623.24; measured m/z 1623.27). G1410 is still methylated on the guanine base (with a visible product ion at m/z 166.07), whereas the C1409 ribose methylation has been lost; an identical pattern was seen for the C-202, 315-A and P2U tlyA strains (not shown).

FIG. 5.

MALDI-TOF mass spectra of RNase A oligonucleotides from the mycobacteria 23S rRNA C1920 region.

(A) The M. tuberculosis H37Rv wild-type rRNA with a fragment at m/z 1301.2, corresponding to methylated AACCp (A1918-C1921); cleavage between C1920 and C1921 has been blocked by the ribose methylation. The 1301.2 Da peak was also observed in rRNA from the other wild-type strains (M. tuberculosis Beijing D3 and the M. smegmatis LR222).

(B) In the M. tuberculosis C-211 mutant rRNA, RNase A cleaves after C1920, and the 1301.2 Da signal is absent. Nucleotide C1920 runs in the unmethylated AACp fragment (A1918-C1920) at m/z 982.2 together with A1912-C1914. The same pattern was seen for the mutant strains M. tuberculosis C-202, C-401 and 315A, and M. smegmatis P2U (data not shown).

(C) The theoretical masses of protonated RNase A fragments from the A1899-U1951 sequence match the empirically measured m/z values to within 0.1 Da in all cases.

FIG. 6.

Tandem mass spectra of the A1918-C1921 oligo from M. tuberculosis H37Rv wild-type rRNA. Backbone cleavage of methylated AACCp (theoretical m/z 1301.22; measured m/z 1301.31) results in unmethylated w1 and z1 ions, and methylated y2 and z2 ions, and this localizes the methyl group to nucleotide C1920. No methyl group is attached to the cytosine base because the a3 ion (at m/z 652.16) has lost unmodified adenine and cytosine nucleobases, and the y3 ion (m/z 750.14) is missing two unmodified cytosine bases. However, a methylated ribose is detected at m/z 111.05 (insert). The same fragmentation patterns, showing C1920 ribose methylation, were also obtained from the other wild-type strains, M. tuberculosis Beijing D3 and M. smegmatis LR222 (not shown).

FIG. 7

Locations of the nucleotides involved in capreomycin resistance within the tertiary structures of the rRNAs. The 16S (top left hand side; dark grey) and 23S (right hand side; light grey) rRNAs are shown within the 70S ribosome crystal structure (Yusupov et al., 2001); the ribosomal proteins have been removed computationally for clarity. The ribosomal A site is indicated by the bound tRNA (dark molecule between the rRNAs); the nucleotides implicated in capreomycin resistance are C1920 (1) in 23S rRNA, and C1409 (2), A1408 (3) and G1491 (4) in 16S rRNA. Enlargement of this region of interest from the same angle, and viewed from above after rotating 90° (upper and lower panels right, respectively). The four nucleotides are located within close proximity to each other at the interface region between the two subunits. The lower right panel shows that 16S rRNA nucleotides A1408, C1409 and G1491 are located within the ribosomal A site, while 23S rRNA C1920 is located towards the P site (Yusupov et al., 2001).

FIG. 8.

Protein sequences of TlyA from Mycobacterium tuberculosis and Mycobacterium smegmatis.

FIG. 9.

(A) Model of the ribosome presenting nucleotides C1409 (16S rRNA in 30S subunit) and C1920 (23S rRNA in 50S subunit). 30S subunit shown in dark grey, 50S subunit in light grey. 2′-O methylation sites are highlighted by elliptical rings.

(B) Close-up of the interface region around nucleotides C1409 (16S rRNA) and C1920 (23S rRNA) involved in capreomycin binding. Distance between the two nucleotides (highlighted by a dashed line) varies between 19.8 and 21.4 Ångstrom, depending on the conformation of the ribosome (data based on Schuwirth et al., 2005). 16S rRNA below (dark), 23S rRNA above (light). 2′-O methylation sites are shown at either end of the dashed line. Relevant nucleotide numbering is presented to provide directionality.

(C) Nucleotides that are at least in part within a 10 Ångstrom radius of 1409C-O-2′ (16S rRNA) or the 2′-O position of 1920C (23S rRNA). nt, nucleotide. In all subfigures the numbers indicate the nucleotide number in E. coli and the corresponding base for said position.

FIG. 10. Hydroxyl radical footprints of capreomycin (Cap) and viomycin (Vio) on the 70S ribosome (left panel) and 50S ribosomal subunit (right panel). Samples were left unmodified (−), or were modified for 10 min with hydroxyl radical without drugs (+), or modified after the addition of 10- or 50-fold excesses of the drugs. The vertical lines indicate modification changes caused by drug binding.

FIG. 11. DMS footprints of capreomycin (Cap) and viomycin (Vio) on the 70S ribosome (left panel) and 50S ribosomal subunit (right panel). Samples were left unmodified (−), or were modified for 10 min with DMS without drugs (+), or modified after the addition of 10- or 50-fold excesses of the drugs. Drug protections were seen only in 70S and not in purified 50S subunits.

FIG. 12. DMS footprints of capreomycin (Cap) and viomycin (Vio) on the 70S ribosome (left panel) and 30S ribosomal subunit (right panel). Samples were left unmodified (−), or were modified for 10 min with DMS without drugs (+), or modified after the addition of 10- or 50-fold excesses of the drugs. Drug protection at nucleotide A1408 was observed only for 70S.

FIG. 13. Footprint effects outside the region of drug contact. Hydroxyl radical footprints of capreomycin (Cap) and viomycin (Vio) on the 70S ribosome (left panel) and 50S ribosomal subunit (right panel). Samples were left unmodified (−), or were modified for 10 min with hydroxyl radical without drugs (+), or modified after the addition of 10- or 50-fold excesses of the drugs. The vertical lines indicate modification changes caused by drug binding.

FIG. 14. Footprint effects outside the region of drug contact. Hydroxyl radical footprints of capreomycin (Cap) and viomycin (Vio) on the 70S ribosome (left panel). No comparable protection effects were seen on the 50S ribosomal subunit (right panel). Samples were left unmodified (−), or were modified for 10 min with hydroxyl radical without drugs (+), or modified after the addition of 10- or 50-fold excesses of the drugs. The vertical lines indicate modification changes caused by drug binding.

FIG. 15. Footprint effects outside the region of drug contact. DMS footprints of capreomycin (Cap) and viomycin (Vio) on the 70S ribosome (left panel) and 30S ribosomal subunit (right panel). Samples were left unmodified (−), or were modified for 10 min with DMS without drugs (+), or modified after the addition of 10- or 50-fold excesses of the drugs. Drug protections at nucleotide A790, A792 and A794 were observed only for 70S.

FIG. 16. Footprint effects outside the region of drug contact. Hydroxyl radical footprints of capreomycin (Cap) and viomycin (Vio) on the 70S ribosome (left panel) and 30S ribosomal subunit (right panel). Samples were left unmodified (−), or were modified for 10 min with hydroxyl radical without drugs (+), or modified after the addition of 10- or 50-fold excesses of the drugs. The vertical lines indicate modification changes caused by drug binding. With this reagent, no effects are seen at the site of drug contact on the 16S rRNA (close to G1410).

The sum of these effects is summarized in FIG. 17 on the crystal structure of the 70S ribosome.

FIG. 17. Summation of the footprint effects illustrated on the crystal structure of the Escherichia coli 70S ribosome. A. Top view of the 30S subunit, showing 16S rRNA (thin, light grey ribbon) is on the left; the 50S subunit containing 23S rRNA (thin, grey ribbon) is on the right. The positions of the A-site tRNA (lower, thick, grey ribbon) and P-site tRNA (upper, thick, grey ribbon) are shown for reference (tRNAs were not included in the footprinting experiments). The ribosomal proteins have been removed for clarity. B. The side view looking in from the A site. Footprint sites are shown in black, and for DMS are at 16S rRNA nucleotides 790, 792, 794 & 1408, and 23S rRNA nucleotides 1889, 1899-1901, 1913, 1918, 1919, 1928, 1937 & 1938. Hydroxyl radical footprints are at 16S rRNA nucleotides 1420, 1426-1428, 1431 & 1438, and 23S rRNA nucleotides 825, 826, 838, 839, 1642-1647, 1660-1661, 1679-1681, 1683-1685, 1692, 1703, 1704, 1708, 1709, 1919, 1920 & 1928-1932. The two rings of 10A radius have their centres at the sites of 2′-methylation on 16S rRNA nucleotide C1409 and 23S rRNA nucleotide C1920.

FIG. 18. Gel autoradiograms of primer extension on the rRNAs to show TlyA methylation. The rRNAs were isolated from E. coli strain DH1 that contained an empty plasmid vector (No tlyA), or contained recombinant tlyA genes from the species indicated. Expression of recombinant genes was induced with IPTG in all cases. Upper panel, primer extension from 23S rRNA nucleotide 1925; lower panel, primer extension from 16S rRNA nucleotide 1412 primer. Samples are in the same order in both autoradiograms. Decreasing the dGTP concentration (indicated by wedges) intensifies reverse transcription termination at nucleotides with 2′-O-methylation. No termination occurs here in the control E. coli strain that lacks tlyA. Reverse transcription was terminated completely at nucleotides A1919 and G1405 by inclusion of ddTTP and ddCTP in the respective extension reactions; unmodified E. coli rRNA was used as the template for the dideoxy-sequencing reactions (lanes C, U, A and G).

FIG. 19. Sensitivity of wild-type streptomycetes to capreomycin. Streptomyces avermitilis MA-4680, Streptomyces coelicolor M145 Streptomyces lividans 1326 and Streptomyces griseus NRRL-B2682 were streaked on SFM agar plates without or with 1 or 5 μg/ml capreomycin.

FIG. 20. Spontaneous capreomycin resistant strains (17 in total) were streaked on SFM agar plates with increasing concentrations of capreomycin (numbers above the plates indicate the concentration in μg/ml). Note the strong pigmentation induced by capreomycin at concentrations above 5 μg/ml.

FIG. 21. Primer extension analysis showing that in mutant CAP9 both C1409 (16S rRNA) and C1920 (23S rRNA) are unmethylated, and that this methylation is restored by the introduction of wild-type copy of tlyA. Control M145 showed the expected normal methylation patterns.

FIG. 22. Resistance of the tlyA in-frame deletion mutant of S. coelicolor. The mutant still grows at 100 μg/ml, while the wild-type strain S. coelicolor M145 is unable to grow at concentrations above 5 μg/ml. Two integrants (Int1 and Int2) which carry the knockout construct pGWS410 after the first recombination event and still have a wild-type copy of tlyA are used as another control.

FIG. 23. Sequences of the mutants CAP9, CAP15 and CAP17

Sequences of the spontaneous mutants CAP9 and CAP15, which carry an insertion of a C residue after nt position 585, and of CAP17, which carries an inserion of a G residue after nt position 481. Inserted nt are in bold face and underlined in the sequences. Derived amino acid sequences up to an including the first stop codon are shown, with changed amino acid sequence underlined.

TABLE 1
M. smegmatis and M. tuberculosis strains used in this study.
The M. tuberculosis 315-A, C-202 and C-211 mutants and the
M. smegmatis P2U mutant have been described previously
(Maus et al., 2005a). The M. tuberculosis Beijing C-401 mutant
was isolated as described in Experimental Procedures.

| Strain | Genotype | MIC | Source/Reference |
|---|---|---|---|
| M. smegmatis | Wild-type | <10 | MLB |
| LR222 | | | |
| M. smegmatis P2U | EZ::TN at tlyA nt 163 | 40 | (Maus et al., 2005a) |
| M. tuberculosis | Wild-type | <10 | MLB |
| H37Rv | | | |
| M. tuberculosis | Wild-type | <10 | MLB |
| Beijing D3 | EZ::TN at tlyA nt. 644 | 20 | (Maus et al., 2005a) |
| M. tuberculosis | C→T at tlyA nt 7: | 40 | (Maus et al., 2005a) |
| 315-A | | | |
| M. tuberculosis | Arg3→STOP | 40 | (Maus et al., 2005a) |
| C-202 | | | |
| M. tuberculosis | C→T at tlyA nt 64: | >160 | This study |
| C-211 | | | |
| M. tuberculosis | Gln22→STOP | | |
| Beijing | | | |
| C-401 | Δ1916 in rrl gene | | |

Abbreviations:
MLB, Mycobacteriology Laboratory Branch, Centers for Disease Control and Prevention;
MIC, minimal inhibitory concentration of capreomycin in μg/ml;
nt, nucleotide mutated in tlyA gene.

TABLE 2
Changes in antibiotic sensitivity in E. coli
upon expression of tlyA.

| | Minimal inhibitory concentrations (μg/ml) | | | |
|---|---|---|---|---|
| Strain | rifampicin | kanamycin | capreomycin | viomycin |
| E. coli DH1/pLJ102 | 4 | 4 | 128 | 128 |
| (TlyA⁻) | | | | |
| E. coli DH1/pSJ102 | 4 | 4 | 32 | 16 |
| (TlyA⁺) | | | | |

Growth of the permeable E. coli strain AS19 was consistent with the above values and the effects of tlyA expression (deviating only in the case of rifampin to which AS19 is hypersensitive with an MIC of 0.1 μg/ml). Expression of the plasmid-encoded copy of the mycobacterial tlyA gene led to complete methylation of the 16S rRNA C1409 and 23S rRNA C1920 riboses in E. coli (not shown). Inactivation of the rlmA^{I} gene (formerly rrmA) that encodes a 23S rRNA G745 N-1 methyltransferase in the E. coli AS19 strain (Liu and Douthwaite, 2002) did not affect the MIC values.

TABLE 3
Primers used for extension analysis with reverse transcriptase to
show the sites of the footprints effects on the M. smegmatis
rRNAs. The E. coli rRNA nucleotide numbering system is
used throughout.

| Oligonucleotide | Complementary to |
|---|---|
| code | rRNA nucleotides |
| SJ21 | 1972-1990, 23S rRNA |
| SJ25 | 1464-1481, 16S rRNA |
| SJ30 | 1741-1761, 23S rRNA |
| SJ31 | 940-962, 23S rRNA |
| SJ33 | 1431-1450, 16S rRNA |
| SJ35 | 821-841, 16S rRNA |

TABLE 4
Sources of tlyA genes that were cloned and expressed in E. coli.

| | | Nucleotide | |
|---|---|---|---|
| | | positions of the | |
| | Gene (acc. | tlyA genes in | Protein (acc. |
| Bacteria | no.) | genomes | no.) |
| Mycobacterium | CP000840 | 3816778 to 3817587 | ABK71205 |
| smegmatis MC2 155 | | | |
| Thermus | AP008226 | 511266 to 511694 | BAD70369 |
| thermophilus HB8 | | | |
| Streptomyces | NC_003888 | 1907225 to 1908040 | NP_626053 |
| coelicolor A3(2) | | | |
| Serpulina | X61684 | No genome sequence | CAA43858 |
| hyodysenteriae | | | |
| Geobacillus | AB126617 | 5120 to 5968 | BAD18315 |
| stearothermophilus | | (partial genome | |
| | | sequence only) | |

TABLE 5
Primers used for PCR of tlyA-genes from
different organisms. Amplification was carried
out in two steps for each tlyA gene: first with
the upstream and downstream primers, and then
with the nested upstream and downstream primers.
NdeI and BglII (or BamHI) restriction-sites
for cleavage and cloning are shown in italics.

| Oligo | Sequence (5′→3′) | Description |
|---|---|---|
| SJ8 | CCG CAT ATG GCA CGG CGA | Nested upstream |
| | GCT CG | primer with NdeI |
| | | site (M. smegmatis) |
| SJ9 | GCT CAA AGA TCT TTG CGG | Nested downstream |
| | CCC TTC CTC G | primer with BglII |
| | | site (M. smegmatis) |
| SJ10 | GTC GAC AAG GGC CAG GTG | Upstream primer |
| | | (M. smegmatis) |
| SJ11 | GTC GCC GAG TAC CTT TTC | Downstream primer |
| | GAC | (M. smegmatis) |
| SJ39 | TAC CCA TAT GCG CCT GGA | Nested upstream |
| | CCG CTA CC | primer with NdeI |
| | | site (T. |
| | | thermophilus) |
| SJ40 | GGA GTA GAT CTA GGG CGC | Nested downstream |
| | CTG AGC C | primer with BglII |
| | | site (T. |
| | | thermophilus) |
| SJ41 | CCC TCC AAA ACG TAC CAG | Upstream primer |
| | GTC CC | (T. thermophilus) |
| SJ42 | CGC CTT AAA CGC CGT GCA | Downstream primer |
| | GAT CG | (T. thermophilus) |
| SJ43 | CCG CAT ATG GCA GGA GTC | Nested upstream |
| | GCA CG | primer with NdeI |
| | | site (S. coelicolor) |
| SJ44 | CGA TTC TAG ATC TAC GCG | Nested downstream |
| | GCC CCT CC | primer with BglII |
| | | site (S. coelicolor) |
| SJ45 | GTA CGA CCG CAG GAG CTG | Upstream primer |
| | AAC C | (S. coelicolor) |
| SJ46 | GGC GAG CAG GAA AAC AGT | Downstream primer |
| | ACG CAC | (S. coelicolor) |
| SJ47 | CTA ATA CAT ATG CGA TTA | Nested upstream |
| | GAT GAA TAT GTG C | primer with NdeI |
| | | site (S. |
| | | hyodysenteriae) |
| SJ51 | CAC ATA AAA TAG AAA AGG | Nested downstream |
| | ATC CAA TAA TAA AAT GAG | primer with BamHI |
| | C | site (S. |
| | | hyodysenteriae) |
| SJ49 | GGA GAT GAA TAT ACT GAA | Upstream primer |
| | ACT TTT GAA TCA G | (S. hyodysenteriae) |
| SJ50 | GGT AAA TGA TGT AGA AGG | Downstream primer |
| | CTT CTA TAA AG | (S. hyodysenteriae) |
| SJ53 | GCT TGA GCA TAT GAA AGG | Nested upstream |
| | GAA AAA AGA ACG | primer with NdeI |
| | | site (G. |
| | | stearothermophilus) |
| SJ54 | GGT AGA TCT GGC GCC GTT | Nested downstream |
| | TTC TTC | primer with BglII |
| | | site (G. |
| | | stearothermophilus) |
| SJ55 | CGC GCA TAT CGT TGA CCG | Upstream primer (G. |
| | C | stearothermophilus) |
| SJ56 | CCT AAG ATG CGC AGT CGC | Downstream primer (G. |
| | G | stearothermophilus) |

TABLE 6
The minimal inhibitory concentrations (MICs) of capreomycin
and viomycin that prevented growth of E. coli DH1 on rich
medium agar (Luria Betani) plates incubated for 22 h at 37° C.
Plates additionally contained 25 μg/ml ampicillin to maintain the
plasmids, and 40 μg/ml IPTG to induce tlyA expression from the
plasmid-encoded lac promoter. n.d., not determined.

| | In vivo methylation | | | |
|---|---|---|---|---|
| Plasmid-encoded tlyA | pattern | | MIC | MIC |
| in the E. coli strain | C1920 | C1409 | capreomycin | viomycin |
| DH1 | 23S rRNA | 16S rRNA | (μg/ml) | (μg/ml) |
| Empty plasmid, no | − | − | 128 | 128 |
| tlyA | | | | |
| M. smegmatis tlyA | + | + | 32 | 16 |
| T. thermophilus tlyA | + | − | 32 | 32 |
| S. coelicolor tlyA | + | + | 64 | 64 |
| S. hyodysenteriae tlyA | + | − | 32 | 32 |
| G. stearothermophilus | + | + | 64 | n.d. |
| tlyA | | | | |

1-48. (canceled)

49. A method for determining whether a compound inhibits growth of a bacterium, the method comprising:
creating a three-dimensional model of a compound,
fitting the three-dimensional model into a space that bridges the 5OS and the 3OS subunits where the helix 44 of the 16S rRNA in the 3OS subunit meets helix 69 of the 23S rRNA in the 5OS subunit (bordered by a 23S rRNA, a 16S rRNA, interbridge B2a, and interbridge B2b) in a three-dimensional model of a 3OS subunit and a 5OS subunit of a ribosome, the fitting comprising establishing interaction between residues of the compound and the subunits in the space,
producing the compound or equivalent thereof,
contacting the compound or equivalent in vitro with the bacterium, and
determining whether growth of the bacterium is at least in part inhibited by the compound.

50. A method for determining whether a compound has antibiotic activity, the method comprising:
providing, to a first bacterium comprising ribosomes having a 3OS subunit and a 5OS subunit, wherein a 23S rRNA in the 50S subunit is not methylated at a nucleotide that corresponds to nucleotide C 1920 in E. coli, a compound of formula I: 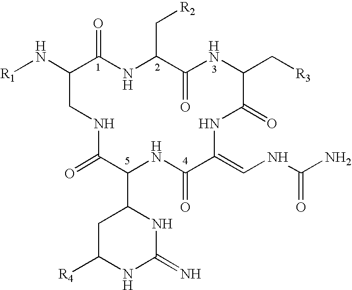
formula (I), wherein R1 and R3 are independently selected from the group consisting of hydrogen, a hydroxy group, an aromatic group, an acyl group, an amino acid, a lysine, an amide-bond forming molecule, a polyketide, a glycosyl group, an alkyl group, a carbohydrate, a halogenated side chain, and a fatty acid, wherein R2 and R4 are independently selected from the group consisting of a hydrogen, a methyl group, a hydroxy group, an acyl group, an amino acid or other amide-bond forming molecule, a fatty acid, a carbohydrate, and a polyketide, and
determining whether the compound inhibits growth of the first bacterium.

51. A method for determining whether a compound is capable of inhibiting growth of a bacterium, the method comprising:
selecting from a collection of compounds a compound that binds in vitro to helix 69 of 23S rRNA, and
providing the compound to a bacterium to determine whether the compound inhibits the growth of the bacterium
so as to determine whether the compound is capable of inhibiting growth of the bacterium.

52. A method for selecting a compound, the method comprising:
determining the stereometrical properties of an antibiotic, and
modifying the properties to fit a binding site of capreomycin bordered by a nucleotide that corresponds to nucleotide C 1920 in E. coli in helix 69 of 23S rRNA so as to select the compound.

53. The method according to claim 49, wherein the bacterium comprises ribosomes wherein a 16S rRNA in the 30S subunit is not methylated at a nucleotide that corresponds to nucleotide C1409 in E. coli.

54. The method according to 51, wherein the compound simultaneously interacts with a 50S ribosomal subunit and a 30S ribosomal subunit of the bacterium.

55. The method according to claim 50, further comprising:
determining whether the compound inhibits growth of a second bacterium comprising ribosomes having a 30S subunit and a 50S subunit, wherein a 23S rRNA in the 50S subunit is methylated at a nucleotide that corresponds to nucleotide C1920 in E. coli.

56. The method according to claim 55, wherein the second bacterium comprises ribosomes wherein a 16S rRNA in the 30S subunit is methylated at a nucleotide that corresponds to nucleotide C1409 in E. coli.

57. A method for determining whether a compound has antibiotic activity, the method comprising:
generating a collection of related compounds through combinatorial biosynthesis of cyclic peptides, and
determining whether a compound from the collection inhibits the growth of a first bacterium that comprises ribosomes having a 3OS subunit and a 50S subunit, wherein a 23S rRNA in the 5OS subunit is not methylated at a nucleotide that corresponds to nucleotide C1920 in E. coli.

58. The method according to claim 57, wherein the combinatorial biosynthesis of cyclic peptides comprises:
providing a first bacterium producing a cyclic peptide antibiotic or a derivative thereof, and
altering the biosynthesis pathway of the a cyclic peptide antibiotic or a derivative thereof.

59. The method according to claim 58, wherein altering the biosynthesis pathway comprises introducing into the bacterium at least one gene coding for a non-ribosomal peptide synthetase (NRPS) from a biosynthesis cluster for cyclic peptide antibiotics from another bacterium species.

60. The method according to claim 49, wherein the first bacterium lacks a functional tlyA gene.

61. A compound obtainable by the method according to claim 49.

62. A composition comprising the compound of claim 61.

63. A medicament for at least in part inhibiting growth of a bacterium, the medicament comprising:
the composition of claim 62 in an amount at least in part capable of inhibiting growth of a bacterium.

64. A pharmaceutical composition comprising:
the composition of claim 62, and
a pharmaceutically acceptable carrier.

65. A method for producing a modified capreomycin or viomycin, the method comprising:
providing capreomycin or viomycin with a hydrophilic group.

66. A method of for at least in part inhibiting translational activity by a ribosome of a bacterium, the method comprising:
contacting the bacterium with the composition of claim 14,
so as to, at least in part, inhibit translational activity by a ribosome of the bacterium.

67. A method for conferring sensitivity to an antibiotic to a bacterium resistant to the antibiotic, the method comprising:
inserting a tlyA gene into the genome of the bacterium.

68. A 2′-O-ribose methyltransferase comprising an isolated protein encoded by a tlyA gene.

69. A cell exogenously provided with the 2′-O-ribose methyltransferase of claim 68.

70. The cell of claim 69, wherein the cell produces an antibiotic.

71. The cell of claim 70, wherein the antibiotic produced by the cell is a cyclic peptide.

72. The cell of claim 71, wherein the cell is not a Mycobacterium.
